(19) United States
(12) Patent Application Publication
(10) Pub. No.: US 2026/0194523 A1
(43) Pub. Date: Jul. 9, 2026

(54) NOVEL BIOMARKER COMBINATION FOR THE DIAGNOSIS, PROGNOSIS, STRATIFICATION AND/OR MONITORING OF A THERAPY, OF A CANCER DISEASE

(71) Applicant: KING FAISAL SPECIALIST HOSPITAL & RESEARCH CENTRE, RIYADH (SA)

(72) Inventors: Hazem GHEBEH, RIYADH (SA); Nasser ALRAJHI, RIYADH (SA); Juamanah MIRZA, RIYADH (SA)

(21) Appl. No.: 19/015,020

(22) Filed: Jan. 9, 2025

(51) Int. Cl. G01N 33/569; G01N 1/34; G01N 1/44; G01N 33/574; G01N 33/58; G01N 33/68

(52) U.S. Cl. CPC G01N 33/56972; G01N 1/34; G01N 1/44; G01N 33/57557; G01N 33/5759; G01N 33/581; G01N 33/6872; G01N 2333/7051; G01N 2333/70585; G01N 2333/70596

(57) ABSTRACT

The present invention relates to a new method for the diagnosis, prognosis, stratification and/or monitoring of a therapy, of a cancer disease in a patient. The method is based on the determination of the level of CD3+ Tumor-Infiltrating Lymphocytes and CD24 expression. The new biomarker combination of the present invention allows diagnosing, prognosing, stratifying and/or monitoring of a therapy of various cancer diseases. Furthermore, provided are diagnostic kits for performing the non-invasive methods of the invention.

The Sequence Listing for this application is labeled “SeqList-07Jan25.xml”, which was created on Jan. 7, 2025 and is 1,690 bytes. The entire content is incorporated herein by reference in its entirety.

### FIELD OF THE INVENTION

The present invention relates to a new method for the diagnosis, prognosis, stratification and/or monitoring of a therapy, of a cancer disease in a patient. The method is based on the determination of the level of CD3+ Tumor-Infiltrating Lymphocytes and CD24 expression. The new biomarker combination of the present invention allows diagnosing, prognosing, stratifying and/or monitoring of a therapy of various cancer diseases. Furthermore, provided are diagnostic kits for performing the methods of the invention.

### BACKGROUND OF THE INVENTION

Cancer remains one of the leading causes of morbidity and mortality worldwide, with millions of new cases diagnosed each year. According to recent statistics, the global incidence of cancer is estimated to be over 18 million new cases annually. Among the various types of cancer, the most common include lung, breast, colorectal, prostate, and stomach cancers. To assess the extent and progression of cancer, tumor staging systems, such as the TNM staging system are widely used.

However, even though classical tumor staging is very useful in assessing the extent and progression of cancer, it still has its pitfalls, such as limited accuracy in predicting patient outcomes and guiding personalized treatment strategies. Biomarkers play a crucial role in overcoming these limitations by providing more precise and individualized information about the tumor's biological behavior. They can help in early detection, monitoring treatment response, and identifying potential therapeutic targets, thereby enhancing the overall effectiveness of cancer management.

Thus, for many aspects of research related to cancer diseases, the identification of specific and sensitive biomarkers suitable for effective diagnosis, prognosis and for therapeutic modalities is of high relevance. Even though the number of potential biomarkers has increased markedly over the last decades, only a few have reached the clinical validation stage, and even fewer are employed as dependable therapeutic targets or diagnostic markers.

Nasopharyngeal cancer (NPC) is a cancer that arises from the nasopharynx that, although less common, still presents a significant health concern, particularly in certain regions. NPC is distinct from other types of head and neck cancer in that EBV plays an essential role in the disease biology. NPC is more common in Southeast China, parts of the Middle East, and North Africa, where most patients present with locally advanced disease. Locally advanced nasopharyngeal carcinoma (LA-NPC), defined as NPC at stage III or IV, is the most common form of NPC in Saudi Arabia. While a large percentage of patients are managed successfully using concurrent radio-chemotherapy (CCRT), a fraction relapse, with some eventually having metastatic disease and dying. Unfortunately, prognostic markers that can identify patients likely to relapse are lacking, especially with the limited use of the traditional TNM staging commonly used in cancer.

Thus, there is a need for novel biomarkers that allow the reliable diagnosis, prognosis, stratification and/or monitoring of a therapy, of a cancer disease. Moreover, there is a need for novel methods for the diagnosis, prognosis, stratification and/or monitoring of a therapy, of a cancer disease in a subject. Moreover, there is a need for novel methods for evaluating the treatment success of a patient suffering from a cancer disease who received a cancer treatment. Moreover, there is a need for a diagnostic kit for performing said methods.

### SUMMARY OF THE INVENTION

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In a first aspect, the present invention relates to a method for the diagnosis, prognosis, stratification and/or monitoring of a therapy, of a cancer disease in a subject, comprising the steps of:
(a) Providing a biological sample from the subject; and
(b) Determining the levels of at least two biomarkers in the biological sample, wherein the at least two biomarkers are CD3+ tumor-infiltrating lymphocytes (TIL) and CD24+ cells;
   wherein levels of the at least two biomarkers in the biological sample from the subject as determined in step (b) that are different from a healthy control or reference values are indicative of the presence of a cancer disease in the subject.

In a preferred embodiment, the method is an ex-vivo and/or in-vitro and/or non-invasive and/or minimally invasive method.

In one embodiment, the biological sample is a sample of a subject comprising a tissue sample or a body liquid sample, such as a blood sample, a serum sample, a plasma sample, a sample of a group of cells from a tumor, a tumor tissue, a urine sample, a lymph fluid sample, a pleural fluid sample, or a brain liquor sample; wherein, preferably, the biological sample is a tissue sample.

In a preferred embodiment, the biological sample is a paraffin-embedded tissue sample, preferably a formalin-fixed paraffin-embedded tissue sample.

In one embodiment, the levels of the at least two biomarkers are assessed by their protein biomarkers, wherein the determining the level of each of the at least two biomarkers involves determining the levels of proteins of the at least two biomarkers in the biological sample.

In one embodiment, the method further comprises determining the level of one or more additional biomarkers in the biological sample, wherein the one or more additional biomarkers are selected from CD44 and markers for epithelial-mesenchymal transition, such as vimentin E-cadherin, N-cadherin, or β-catenin; preferably selected from CD44 and vimentin; even more preferably CD44.

In one embodiment, determined level(s) of the one or more additional biomarkers in the biological sample from the subject that is/are different from a healthy control or reference value(s) may be further indicative of the presence of an aggressive cancer disease in the subject.

In one embodiment, if the determining of the level of in the biological sample leads to the identification of a CD44+^{high} phenotype, this phenotype may be further indicative of an aggressive cancer disease in the subject (i.e. a cancer disease that is not responsive to standard treatment and/or progresses faster than usual for this type of disease).

In one embodiment, if the determining of the level of in the biological sample leads to the identification of vimentin^{high} phenotype, this phenotype may be further indicative of an aggressive cancer disease in the subject (i.e. a cancer disease that is not responsive to standard treatment and/or progresses faster than usual for this type of disease).

In one embodiment, if the determining of the level of in the biological sample leads to the identification of E-cadherin^{low} phenotype, this phenotype may be further indicative of an aggressive cancer disease in the subject (i.e. a cancer disease that is not responsive to standard treatment and/or progresses faster than usual for this type of disease).

In one embodiment, if the determining of the level of in the biological sample leads to the identification of a phenotype with upregulated β-catenin expression, this phenotype may be further indicative of an aggressive cancer disease in the subject (i.e. a cancer disease that is not responsive to standard treatment and/or progresses faster than usual for this type of disease).

In one embodiment, if the determining of the level of in the biological sample leads to the identification of a phenotype with upregulated N-cadherin expression, this phenotype may be further indicative of an aggressive cancer disease in the subject (i.e. a cancer disease that is not responsive to standard treatment and/or progresses faster than usual for this type of disease).

In one embodiment, the method is a screening method for establishing a first diagnosis of cancer in the subject.

In one embodiment, if the determining of the levels of the at least two biomarkers in the biological sample results in a CD24+^{high}/CD3+ TIL^{low} phenotype, the subject is prognosed with a high risk of disease progression.

In one embodiment, when the subject is prognosed with a high risk of disease progression, a treatment plan for said patient is modified; wherein, optionally, said modification of said treatment plan comprises one or more of: changing the medication, optionally to investigational agent(s), and intensifying the treatment, such as by increasing the treatment doses and/or adjusting the dosing regimen in order to improve the survival of said patient.

In one embodiment adjusting the dosing regimen comprises one or more of: increasing the frequency of the medication, increasing the overall doses of a medication and/or increasing the treatment duration.

In one embodiment, changing the medication comprises one or more of: the use of a combination of one or more (additional) therapeutic agent(s), the use of more aggressive therapeutic agent(s) and/or the use of investigational therapeutic agent(s).

In one embodiment, the cancer disease is a cancer selected from a group comprising nasopharyngeal cancer, head and neck cancer, hepatocellular cancer, breast cancer, triple-negative breast cancer, colorectal cancer, gastric cancer, lymphomas including non-Hodgkin lymphomas such as Burkitt lymphomas, gliomas, melanomas, small-cell and non-small cell lung cancer, cholangiocarcinoma, renal cell cancer, bladder cancer, uterine cancer, epithelial ovarian cancer, prostate cancer, and pancreatic cancer; wherein, preferably, the cancer is nasopharyngeal cancer, head and neck cancer, or hepatocellular cancer; even more preferably the cancer is nasopharyngeal cancer; most preferably the cancer is locally advanced nasopharyngeal cancer.

In one embodiment, the biomarker is detected using one or more binding agents or antigen-binding peptides, such as antibodies, wherein, preferably, the biomarker is detected by immunohistochemistry, western blot, ELISA, Proximity Extension Assay, or mass spectrometry; wherein, more preferably the biomarker is detected by immunohistochemistry.

In one embodiment, one or more of the least two biomarkers is detected by immunohistochemistry by a method comprising the steps of:
(i) Providing a formalin-fixed paraffin-embedded (FFPE) tissue sample derived from the subject;
(ii) Dewaxing the FFPE tissue sample in xylene and rehydrating it using alcohol/water gradients;
(iii) Performing antigen retrieval using an antigen retrieval solution in a decloaking chamber pressure cooker;
(iv) Optionally, quenching endogenous peroxidase and/or blocking endogenous biotin;
(v) Incubating the sample with one or more primary antibodies that are specific for the one or more of the at least two biomarkers, preferably overnight at about 2-10° C., more preferably at about 4° C., wherein further preferably, the one or more primary antibodies are diluted in 1% BSA;
(vi) Incubating the sample using one or more secondary antibodies that are specific for either of said one or more primary antibodies, wherein said one or more secondary antibodies are conjugated with a reporter enzyme; wherein, optionally, if more than one secondary antibody is used, incubating the sample with the one or more secondary antibodies may be performed either simultaneously or subsequently;
(vii) Detection of the reporter enzyme conjugated to said secondary antibody, wherein, optionally, if more than one primary antibody and/or more than one secondary antibody is used, detection of said secondary antibodies may be performed either simultaneously or subsequently.

In one embodiment, the method further comprises one or more washing steps using a washing buffer, such as TBST, between steps (iv) and (v); and/or between steps (v) and (vi); and/or between steps (vi) and (vii); and/or between the detection of the different secondary antibodies in step (vii); and/or
wherein the antigen retrieval solution of step (iii) is Tris-EDTA with a pH of about 9; and/or
wherein antigen retrieval in step (iii) is performed at a temperature in a range of about 90° C. to about 150° C., preferably in a range of about 110° C. to about 130° C., more preferably at about 121° C., for a time period in a range of about 1 min to 15 min, preferably in a range of about 5 min to about 10 min, more preferably for about 7 min.

In one embodiment, the secondary antibody is diluted in 1% BSA.

In one embodiment, step (vi) is performed at room temperature for a time period in a range of about 10 min to about 2 h, preferably in a range of about 15 min to about 45 min, more preferably in a range of about 20 min to about 30 min, most preferably for about 30 min.

In one embodiment, the conjugated reporter enzyme is selected from a group comprising Alkaline Phosphatase (AP), Horseradish Peroxidase (HRP), β-Galactosidase, Glucose Oxidase, Acid Phosphatase; preferably the conjugated reporter enzyme is Alkaline Phosphatase (AP) or Horseradish Peroxidase (HRP).

In one preferred embodiment, antigen expression, preferably CD24 expression, is assessed using the H-score system.

In one embodiment, CD24 is detected using a binding agent or antigen-binding peptide that specifically binds to CD24, wherein the CD24 comprises or consists of an amino acid sequence which is at least 90% identical, more preferably at least 95% identical, even more preferably at least 98% identical, even more preferably at least 99% identical, optionally identical, to an amino acid of SEQ ID No. 1;
wherein, preferably, said binding agent or antigen-binding peptide binds to a sequence according to positions 46-48 of SEQ ID No. 1.

In one embodiment, the binding agent or antigen-binding peptide that specifically binds to CD24 is selected from a group comprising: anti-CD24 monoclonal antibody clone: ML5, anti-CD24 monoclonal antibody clone: M1/69, anti-CD24 monoclonal antibody clone: SN3b, anti-CD24 monoclonal antibody clone: SN3, anti-CD24 monoclonal antibody clone: SWA11, and anti-CD24 monoclonal antibody clone: ALB9; preferably selected from anti-CD24 monoclonal antibody clone: ML5 and anti-CD24 monoclonal antibody clone: M1/69; even more preferably anti-CD24 monoclonal antibody clone: ML5.

In one embodiment, said subject is a mammal, such as a mouse, a rat, a guinea pig, a rabbit, a cat, a dog, a monkey, or a human, preferably a human.

In one aspect, the present invention relates to a method for evaluating the treatment success of a patient suffering from a cancer disease who received a cancer treatment, comprising the steps of:
(a) Providing a biological sample from the subject;
(b) Determining the levels of at least two biomarkers in the biological sample, wherein the at least two biomarkers are CD3+ tumor-infiltrating lymphocytes (TIL) and CD24+ cells; and
(c) Comparing the levels of said at least two biomarkers as determined in (b) with a reference sample or reference values,
   wherein a decrease or an increase of the levels of the at least two biomarkers in the biological sample from said subject compared to said reference sample or reference values are indicative of the patient's response to said treatment.

In a preferred embodiment, the method is an ex-vivo and/or in-vitro and/or non-invasive and/or minimally invasive method.

In one embodiment, said reference values correspond to the levels of the at least two biomarkers in a provided biological sample obtained from said patient before receiving said treatment.

In one embodiment, a high CD24 level and/or a low level of CD3+ TIL, is indicative of an insufficient response by the patient to said treatment.

In one embodiment, the patient is a mammal, such as a mouse, a rat, a guinea pig, a rabbit, a cat, a dog, a monkey, or a human, preferably a human.

In one embodiment, the cancer disease is a cancer selected from a group comprising nasopharyngeal cancer, head and neck cancer, hepatocellular cancer, breast cancer, triple-negative breast cancer, colorectal cancer, gastric cancer, lymphomas including non-Hodgkin lymphomas such as Burkitt lymphomas, gliomas, melanomas, small-cell and non-small cell lung cancer, cholangiocarcinoma, renal cell cancer, bladder cancer, uterine cancer, epithelial ovarian cancer, prostate cancer, and pancreatic cancer; wherein, preferably, the cancer is nasopharyngeal cancer, head and neck cancer, or hepatocellular cancer; even more preferably the cancer is nasopharyngeal cancer; most preferably the cancer is locally advanced nasopharyngeal cancer.

In one embodiment, the biomarker is detected using one or more binding agents or antigen-binding peptides, such as antibodies, preferably wherein the biomarker is detected by immunohistochemistry, western blot, ELISA, Proximity Extension Assay, or mass spectrometry; wherein, more preferably the biomarker is detected by immunohistochemistry.

In one embodiment, one or more of the least two biomarkers is detected by immunohistochemistry by a method comprising the steps of:
(i) Providing a formalin-fixed paraffin-embedded (FFPE) tissue sample derived from the subject;
(ii) Dewaxing the FFPE tissue sample in xylene and rehydrating it using alcohol/water gradients;
(iii) Performing antigen retrieval using an antigen retrieval solution in a decloaking chamber pressure cooker;
(iv) Optionally, quenching endogenous peroxidase and/or blocking endogenous biotin;
(v) Incubating the sample with one or more primary antibodies that are specific for the one or more of the at least two biomarkers, preferably overnight at about 2-10° C., more preferably at about 4° C., wherein further preferably, the one or more primary antibodies are diluted in 1% BSA;
(vi) Incubating the sample using one or more secondary antibodies that are specific for either of said one or more primary antibodies, wherein said one or more secondary antibodies are conjugated with a reporter enzyme; wherein, optionally, if more than one secondary antibody is used, incubating the sample with the one or more secondary antibodies may be performed either simultaneously or subsequently;
(vii) Detection of the reporter enzyme conjugated to said secondary antibody, wherein, optionally, if more than one primary antibody and/or more than one secondary antibody is used, detection of said secondary antibodies may be performed either simultaneously or subsequently.

In one embodiment, the method further comprises one or more washing steps using a washing buffer, such as TBST, between steps (iv) and (v); and/or between steps (v) and (vi); and/or between steps (vi) and (vii); and/or between the detection of the different secondary antibodies in step (vii); and/or
wherein the antigen retrieval solution of step (iii) is Tris-EDTA with a pH of about 9; and/or
wherein antigen retrieval in step (iii) is performed at a temperature in a range of about 90° C. to about 150° C., preferably in a range of about 110° C. to about 130° C., more preferably at about 121° C., for a time period in a range of about 1 min to 15 min, preferably in a range of about 5 min to about 10 min, more preferably for about 7 min.

In one embodiment, the secondary antibody is diluted in 1% BSA.

In one embodiment, step (vi) is performed at room temperature for a time period in a range of about 10 min to about 2 h, preferably in a range of about 15 min to about 45 min, more preferably in a range of about 20 min to about 30 min, most preferably for about 30 min.

In one embodiment, the conjugated reporter enzyme is selected from a group comprising Alkaline Phosphatase (AP), Horseradish Peroxidase (HRP), β-Galactosidase, Glucose Oxidase, Acid Phosphatase; preferably the conjugated reporter enzyme is Alkaline Phosphatase (AP) or Horseradish Peroxidase (HRP).

In one preferred embodiment, antigen expression, preferably CD24 expression, is assessed using the H-score system.

In one embodiment, CD24 is detected using a binding agent or antigen-binding peptide that specifically binds to CD24, wherein the CD24 comprises or consists of an amino acid sequence which is at least 90% identical, more preferably at least 95% identical, even more preferably at least 98% identical, even more preferably at least 99% identical, optionally identical, to an amino acid of SEQ ID No. 1;
wherein, preferably, said binding agent or antigen-binding peptide binds to a sequence according to positions 46-48 of SEQ ID No. 1.

In one embodiment, the binding agent or antigen-binding peptide that specifically binds to CD24 is selected from a group comprising: anti-CD24 monoclonal antibody clone: ML5, anti-CD24 monoclonal antibody clone: M1/69, anti-CD24 monoclonal antibody clone: SN3b, anti-CD24 monoclonal antibody clone: SN3, anti-CD24 monoclonal antibody clone: SWA11, and anti-CD24 monoclonal antibody clone: ALB9; preferably selected from anti-CD24 monoclonal antibody clone: ML5 and anti-CD24 monoclonal antibody clone: M1/69; even more preferably anti-CD24 monoclonal antibody clone: ML5.

In one embodiment, said subject is a mammal, such as a mouse, a rat, a guinea pig, a rabbit, a cat, a dog, a monkey, or a human, preferably a human.

In one aspect, the present invention relates to a diagnostic kit for performing a method as defined herein, the kit comprising a combination of at least two antibodies, derivatives, or antigenic fragments thereof, for the detection of any of CD24 and CD3.

In one embodiment, the kit further comprises additional components selected from a group comprising: visualization systems, blocking agents; chromogenic solutions, wash buffers; instructions for suitable operational parameters; secondary antibodies optionally conjugated with reporter enzymes, wherein said secondary antibodies are specific for at least one of said at least two antibodies; antigen retrieval solutions; detection kits for the detection of said conjugated reporter enzymes, and standard or control information so that the test sample can be compared with the control information standard.

In one aspect, the present invention relates to a method for the diagnosis, prognosis, stratification and/or monitoring of a therapy, of a cancer disease in a subject, comprising determining the levels of at least two biomarkers in a biological sample obtained from the subject, wherein the at least two biomarkers are CD3+ tumor-infiltrating lymphocytes (TIL) and CD24+ cells;
wherein levels of the at least two biomarkers in the biological sample from the subject as determined that are different from a healthy control or reference values are indicative of the presence of a cancer disease in the subject.

According to this aspect, the cancer, the subject, the biomarkers, and the determination of said biomarkers are as defined herein.

In one aspect, the present invention relates to a method for evaluating the treatment success of a patient suffering from a cancer disease who received a cancer treatment, comprising the steps of:
(a) Determining the levels of at least two biomarkers in a biological sample obtained from the subject, wherein the at least two biomarkers are CD3+ tumor-infiltrating lymphocytes (TIL) and CD24+ cells; and
(b) Comparing the levels of said at least two biomarkers as determined with a reference sample or reference values,
   wherein a decrease or an increase of the levels of the at least two biomarkers in the biological sample from said subject compared to said reference sample or reference values are indicative of the patient's response to said treatment.

According to this aspect, the cancer, the subject, the biomarkers, and the determination of said biomarkers are as defined herein.

In one aspect, the present invention relates to CD3+ tumor-infiltrating lymphocytes (TIL) and CD24+ cells for use in a method for the diagnosis, prognosis, stratification and/or monitoring of a therapy, of a cancer disease in a subject. Such use typically comprises determining the levels of at least CD3+ tumor-infiltrating lymphocytes (TIL) and CD24+ cells in a biological sample from the subject.

According to this aspect, the cancer and the subject are as defined herein.

In one aspect, the present invention relates to a method of detecting an antigen, preferably CD24, in a biological sample derived from a subject, the method comprising the steps of:
(i) Providing a formalin-fixed paraffin-embedded (FFPE) tissue sample derived from the subject;
(ii) Dewaxing the FFPE tissue sample in xylene and rehydrating it using alcohol/water gradients;
(iii) Performing antigen retrieval using an antigen retrieval solution in a decloaking chamber pressure cooker;
(iv) Optionally, quenching endogenous peroxidase and/or blocking endogenous biotin;
(v) Incubating the sample with one or more primary antibodies that are specific for the one or more of the at least two biomarkers, preferably overnight at about 2-10° C., more preferably at about 4° C., wherein further preferably, the one or more primary antibodies are diluted in 1% BSA;
(vi) Incubating the sample using one or more secondary antibodies that are specific for either of said one or more primary antibodies, wherein said one or more secondary antibodies are conjugated with a reporter enzyme; wherein, optionally, if more than one secondary antibody is used, incubating the sample with the one or more secondary antibodies may be performed either simultaneously or subsequently;
(vii) Detection of the reporter enzyme conjugated to said secondary antibody, wherein, optionally, if more than one primary antibody and/or more than one secondary antibody is used, detection of said secondary antibodies may be performed either simultaneously or subsequently.

In one embodiment, the method further comprises one or more washing steps using a washing buffer, such as TBST, between steps (iv) and (v); and/or between steps (v) and (vi); and/or between steps (vi) and (vii); and/or between the detection of the different secondary antibodies in step (vii); and/or
wherein the antigen retrieval solution of step (iii) is Tris-EDTA with a pH of about 9; and/or
wherein antigen retrieval in step (iii) is performed at a temperature in a range of about 90° C. to about 150° C., preferably in a range of about 110° C. to about 130° C., more preferably at about 121° C., for a time period in a range of about 1 min to 15 min, preferably in a range of about 5 min to about 10 min, more preferably for about 7 min.

In one embodiment, the secondary antibody is diluted in 1% BSA.

In one embodiment, step (vi) is performed at room temperature for a time period in a range of about 10 min to about 2 h, preferably in a range of about 15 min to about 45 min, more preferably in a range of about 20 min to about 30 min, most preferably for about 30 min.

In one embodiment, the conjugated reporter enzyme is selected from a group comprising Alkaline Phosphatase (AP), Horseradish Peroxidase (HRP), β-Galactosidase, Glucose Oxidase, Acid Phosphatase; preferably the conjugated reporter enzyme is Alkaline Phosphatase (AP) or Horseradish Peroxidase (HRP).

In one preferred embodiment, antigen expression, preferably CD24 expression, is assessed using the H-score system.

In one embodiment, CD24 is detected using a binding agent or antigen-binding peptide that specifically binds to CD24, wherein the CD24 comprises or consists of an amino acid sequence which is at least 90% identical, more preferably at least 95% identical, even more preferably at least 98% identical, even more preferably at least 99% identical, optionally identical, to an amino acid of SEQ ID No. 1;
wherein, preferably, said binding agent or antigen-binding peptide binds to a sequence according to positions 46-48 of SEQ ID No. 1.

In one embodiment, the binding agent or antigen-binding peptide that specifically binds to CD24 is selected from a group comprising: anti-CD24 monoclonal antibody clone: ML5, anti-CD24 monoclonal antibody clone: M1/69, anti-CD24 monoclonal antibody clone: SN3b, anti-CD24 monoclonal antibody clone: SN3, anti-CD24 monoclonal antibody clone: SWA11, and anti-CD24 monoclonal antibody clone: ALB9; preferably selected from anti-CD24 monoclonal antibody clone: ML5 and anti-CD24 monoclonal antibody clone: M1/69; even more preferably anti-CD24 monoclonal antibody clone: ML5.

### DETAILED DESCRIPTION

The term “subject”, as used herein, relates to a mammal, such as a mouse, a rat, a guinea pig, a rabbit, a cat, a dog, a monkey, or a human, preferably a human.

The term “patient”, as used herein, relates to a mammal, such as a mouse, a rat, a guinea pig, a rabbit, a cat, a dog, a monkey, or a human, preferably a human.

In a particularly preferred embodiment, the subject is a mammal, such as a mouse, a rat, a guinea pig, a rabbit, a cat, a dog, a monkey, or a human, preferably a human, such as a human patient, more preferably a human patient suffering from a disease, such as cancer, and in need of a therapy, even more preferably a human patient suffering from nasopharyngeal cancer and in need of a therapy, most preferably a human patient suffering from locally advanced nasopharyngeal cancer and in need of a therapy.

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, 10%, and for example 5%. As will be appreciated by the person of ordinary skill, the specific such deviation for a numerical value for a given technical effect will depend on the nature of the technical effect.

For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

The term “CD24”, as used herein, refers to a cell surface protein that is involved in cell adhesion and signaling. CD24 may also be referred to as “signal transducer CD24” or “heat stable antigen CD24”. CD24 is a glycosylphosphatidylinositol (GPI)-anchored protein expressed on the surface of various cell types, including hematopoietic cells, neuronal cells, and epithelial cells. It plays a role in the regulation of immune responses and cell proliferation. The term “CD24 positive”, as used herein, refers to cells that express the CD24 protein on their surface. These cells can be identified and characterized based on the presence of CD24, which can be detected using specific antibodies in techniques such as immunohistochemistry or flow cytometry. The level of CD24 is considered high (also referred to as “CD24+^{high}” or “CD24+^{hi}”), when CD24 is expressed by more than 10% of cells, such as cancer cells, preferably more than 20% of cells, such as cancer cells, more preferably more than 30% of cells, such as cancer cells; wherein CD24 is expressed in the membrane, cytoplasm and/or the nucleus of cells, such as cancer cells.

In one embodiment, the terms “level of CD24”, “CD24+”, and the like, refer to the level of CD24 in the membrane of cells.

In one embodiment, the terms “level of CD24”, “CD24+”, and the like, refer to the level of CD24 in the cytoplasm of cells.

In one embodiment, the terms “level of CD24”, “CD24+”, and the like, refer to the level of CD24 in the nucleus of cells.

The term “CD3”, as used herein, refers to a protein complex and cell surface marker that is expressed on T cells. CD3 is composed of several subunits and is an essential component of the T cell receptor (TCR) complex, playing a critical role in T cell activation, signal transduction, and immune response. CD3 is commonly used as a marker to identify and study T cells in various immunological assays and research. The term “a CD3+ cell” or “a CD3 positive cell”, and the like, refers to a cell, which expresses said CD3 protein complex.

The term “tumor-infiltrating lymphocyte”, also referred to as “TIL”, as used herein, refers to a type of immune cell, specifically a lymphocyte, that has migrated from the bloodstream into a tumor. These cells are part of the body's immune response and can include various subsets such as T cells, B cells, and natural killer (NK) cells. Tumor-infiltrating lymphocytes are often analyzed to understand the immune environment within tumors and their role in cancer progression and response to therapy.

The term “CD3+ TIL”, as used herein, refers to tumor-infiltrating lymphocytes (TILs) that express the CD3 marker on their surface. CD3+ TILs are a subset of immune cells, specifically T cells, that have migrated into the tumor microenvironment and are involved in the body's immune response against cancer cells. In the present application, the level of CD3+ TIL is considered low (also referred to as “CD3+ TIL¹-”), if the CD3+ TIL score of the tumor section is 1 or 2, wherein the CD3+ TIL score of 1 is defined as CD3+ TIL occupying from about 0 to about 10% of the tumor section, and wherein CD3+ TIL score of 2 is defined as CD3+ TIL occupying more than about 10% to about 40% of the tumor section.

The terms “diagnosis” or “diagnostic”, or similar expressions, as used herein, refer to identifying the presence or absence, or identifying the nature of a pathologic condition in a subject. Diagnostic methods differ in their sensitivity and specificity. The “sensitivity” of a diagnostic assay is a measure of the percentage of diseased individuals who test positive (percent of “true positives”). Diseased individuals not detected by the assay are “false negatives.” Subjects, who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The term “prognosis”, as used herein, refers to a forecast as to the probable outcome of a disease as well as the prospect of recovery from a disease as indicated by the nature and symptoms of the case. Accordingly, a negative or poor prognosis is defined by a lower post-treatment survival term or survival rate. Conversely, a positive or good prognosis is defined by an elevated post-treatment survival term or survival rate. Usually, prognosis is provided as the time of progression free survival, metastasis free survival and/or overall survival.

The term “stratification” or “stratifying”, as used herein, refers to the division of a patient population into patient subpopulations on the basis of specified criteria. More particularly, it refers to the division of a cohort of subjects or patients into at least two groups on the basis of specific criteria, which in the context of the present invention comprise or consist of the determined levels of the biomarker of the panel of the present invention. The method according to the invention renders possible decisions for the treatment and therapy of the patient, whether it is the hospitalization of the patient, the use, effect and/or dosage of one or more drugs, a therapeutic measure or the monitoring of a course of the disease and the course of therapy or etiology or classification of a disease, e.g., into a new or existing subtype or the differentiation of diseases and the patients thereof. Particularly with regard to nasopharyngeal cancer, “stratification” means in this context a classification of a nasopharyngeal cancer as early-stage nasopharyngeal cancer, locally advanced nasopharyngeal cancer, or locally advanced nasopharyngeal cancer with a negative or poor prognosis.

The term “monitoring a therapy”, as used herein, means to observe disease progression in a subject who receives a therapy, such as cancer therapy. In other words, the subject during the therapy is regularly monitored for the effect of the applied therapy, which allows the medical practitioner to estimate at an early stage during the therapy whether the prescribed treatment is effective or not, and, therefore, to continue, adjust or end the treatment regime accordingly.

The term “healthy control”, as used herein, refers to an individual or group of individuals who do not have the disease or condition being studied and are used as a baseline to compare against those who do have the disease or condition.

The term “reference value”, as used herein, refers to a value obtained by measuring a reference sample that is used for comparison purposes in clinical and laboratory settings to interpret test results. In preferred embodiments a reference value shall correspond to a sample obtained from a healthy individual or obtained from a tumor-adjacent tissue from a subject suffering from a tumor disease. In certain embodiments the reference value may also be derived from a sample obtained from the same subject at an earlier time point.

In preferred embodiments, the terms “increase” or “decrease” of a level of a biomarker in a sample, as used herein, refers to a significant increase or decrease, respectively, when compared to a control or reference values.

In a preferred embodiment the method of the herein disclosed invention is a non-invasive, minimally invasive, ex-vivo or in-vitro method. If the herein described diagnostic methods are non-invasive, the term “providing a biological” sample shall preferably not be interpreted to include a surgical procedure conducted at the subject.

The terms “cancer” and “cancer cells”, as used herein, refer to any cells that exhibit uncontrolled growth in a tissue or organ of a multicellular organism. Particularly preferred cancers in context of the present invention are selected from the group comprising nasopharyngeal cancer, head and neck cancer, hepatocellular cancer, breast cancer, triple-negative breast cancer, colorectal cancer, gastric cancer, lymphomas including non-Hodgkin lymphomas (e.g. Burkitt lymphomas), gliomas, melanomas, small-cell and non-small cell lung cancer, hepatocellular cancer, cholangiocarcinoma, renal cell cancer, bladder cancer, uterine cancer, epithelial ovarian cancer, prostate cancer, and pancreatic cancer; wherein, preferably, the cancer is nasopharyngeal cancer, head and neck cancer, or hepatocellular cancer; even more preferably the cancer is nasopharyngeal cancer; most preferably the cancer is locally advanced nasopharyngeal cancer.

The term “nasopharyngeal cancer”, also referred to as NPC, as used herein, refers to a cancer that arises from the nasopharynx that, although less common, still presents a significant health concern, particularly in certain regions. NPC is distinct from other types of head and neck cancer in that EBV (Epstein-Barr Virus) plays an essential role in the disease biology, especially the undifferentiated type of NPC. NPC is more common in Southeast China, parts of the Middle East, and North Africa. The term “locally advanced nasopharyngeal carcinoma (LA-NPC)”, as used herein, refers to NPC at stage III or IV, and is the most common form of NPC in Saudi Arabia. While a large percentage of patients are managed successfully using concurrent radio-chemotherapy (CCRT), a fraction relapse, with some eventually having metastatic disease and dying.

The term “aggressive cancer disease” or “aggressive form of a cancer disease”, as used herein, refers to a type of cancer that is not responsive to standard treatment and/or progresses faster than usual for this type of disease. This includes cancers that exhibit rapid growth, high metastatic potential, resistance to multiple lines of therapy, and a tendency to recur quickly after treatment. Additionally, aggressive cancer diseases often have poor prognosis and may require more intensive and novel therapeutic approaches.

The term “high risk of disease progression”, as used herein, refers to a clinical assessment indicating that an individual has a significantly increased likelihood of experiencing a worsening or advancement of their disease. This determination is based on various factors, including but not limited to, clinical symptoms, biomarker levels, genetic predispositions, imaging results, and other diagnostic criteria. Individuals identified as having a high risk of disease progression may require more intensive monitoring, treatment, or intervention to manage their condition effectively. Cancer subpopulations having a high risk of disease progression, show a correlation with decreased disease free survival (DSF) and/or metastasis free survival (MSF) and/or overall survival (OS).

The term “biological sample”, as used herein, refers to a sample that was obtained and may be assayed for the at least two biomarkers as disclosed with the present invention, or their gene expression. The biological sample can include a body liquid sample, such as a blood sample, a serum sample, a plasma sample, a sample of a group of cells from a tumor, a tumor tissue, a urine sample, a lymph fluid sample, a pleural fluid sample, or a brain liquor sample, or a tissue sample such as a sample from a tissue biopsy, and the like. In some embodiments, the sample is a tissue sample, for example, a tumor tissue sample, and may be fresh, frozen, or archival paraffin embedded tissue. Preferred samples for the purposes of the present invention are tissue samples.

The terms “biomarker” or “marker”, as used herein refers to an organic biomolecule, particularly a polypeptide, or a cell expressing an organic biomolecule, particularly a polypeptide, which is differentially present in a sample taken from subjects having a certain condition as compared to a comparable sample taken from subjects who do not have said condition (e.g., negative diagnosis, normal or healthy subject, or non-cancer patients, depending on whether the patient is tested for cancer, or metastatic cancer).

The term “determining the level of” a biomarker in a sample, control or reference, as described herein shall refer to the quantification of the presence of said biomarker in the tested sample. For example, number or proportion of cells expressing a certain biomolecule may be assessed. Also, the concentration of the biomarker in said sample may be directly quantified via measuring the amount of protein/polypeptide/polysaccharide as present in the tested sample. Moreover it is possible to quantify the amount of biomarker indirectly via assessing the gene expression of the encoding gene of the biomarker, for example by quantification of the expressed mRNA encoding for the respective biomarker protein. The present invention shall not be restricted to any particular method for determining the level of a given biomarker but shall encompass all means that allow for a quantification, and/or estimation, of the level of said biomarker, either directly or indirectly. The term “Level”, as used herein, is therefore a parameter describing the absolute amount of a biomarker in a given sample; or alternatively “level” pertains to the relative amounts, and preferably to the concentration of said biomarker in the tested sample, for example mol/l, g/l, g/mol etc.

The term “paraffin-embedded tissue sample”, as used herein, refers to a biological tissue specimen that has been preserved and embedded in paraffin wax to maintain its structural integrity for histological examination and analysis.

The term “formalin-fixed paraffin-embedded tissue sample”, as used herein, refers to a biological tissue specimen that has been preserved by fixation in formalin and subsequently embedded in paraffin wax, a common method used to prepare tissue samples to maintain its structural integrity for histological examination and analysis.

The term “CD44”, as used herein, refers to a cell surface glycoprotein involved in cell-cell interactions, cell adhesion, and migration. CD44 is commonly used as a marker in cancer research and is associated with tumor progression and metastasis.

In the present application, the level of CD44+ is considered high (also referred to as “CD44+^{high}” when CD44 is expressed membranously in 70% of cancer cells in a tumor section.

The term “Vimentin”, as used herein, refers to a type of intermediate filament protein that is expressed in mesenchymal cells. It is involved in maintaining cell integrity and providing resistance against stress. Vimentin serves as a marker in epithelial-to-mesenchymal transition (EMT), indicating the process where epithelial cells acquire mesenchymal, fibroblast-like properties and show reduced intercellular adhesion and increased motility.

In the present application, the level of vimentin is considered high (also referred to as “vimentin^{high}”), if vimentin is upregulated in ≥10% of cancer cells in a tumor section.

The term “E-cadherin”, as used herein, refers to a protein that is a cell adhesion molecule, playing a crucial role in maintaining epithelial cell layer integrity and tissue architecture. It is a type of cadherin involved in cell-cell adhesion. E-Cadherin serves as a marker in epithelial-to-mesenchymal transition (EMT), where its downregulation is often associated with the loss of epithelial characteristics and the gain of mesenchymal traits, contributing to increased cell motility and invasiveness.

In the present application, the level of E-cadherin is considered high (also referred to as “E-cadherin^{low}”), if E-cadherin is expressed in ≤70% of cancer cells in a tumor section.

The term “N-Cadherin”, as used herein, refers to a type of cadherin, which is a class of type-1 transmembrane proteins involved in cell-cell adhesion. N-Cadherin is specifically known for its role in neural development and is also implicated in cancer progression and metastasis.

The term “β-Catenin”, as used herein, refers to a protein that is part of the cadherin protein complex and plays a crucial role in the regulation of cell-cell adhesion and gene transcription. β-Catenin is also a key component of the Wnt signaling pathway, which is important in embryonic development and cancer.

The term “investigational agent”, as used herein, refers to a substance or compound that is being tested in clinical trials to evaluate its safety, efficacy, or mechanism of action in treating a specific disease or condition. This term encompasses a wide range of potential therapeutic entities, including small molecules, biologics, vaccines, gene therapies, and other novel therapeutic approaches. Investigational agents are typically in the experimental stage and have not yet received approval from regulatory authorities such as the U.S. Food and Drug Administration (FDA) or the European Medicines Agency (EMA) for general medical use. They are subject to rigorous testing in preclinical studies and multiple phases of clinical trials to determine their therapeutic potential, optimal dosing, side effect profile, and overall benefit-risk ratio.

The term “intensifying the treatment”, as used herein, refers to the process of increasing the strength, dosage, frequency, or combination of therapeutic interventions to enhance the efficacy of a treatment regimen. This may involve adjusting the current treatment plan to achieve better clinical outcomes, address suboptimal responses, or manage disease progression. Intensifying the treatment can include the addition of new therapeutic agents, increasing the dose of existing medications, shortening the intervals between treatments, or employing more aggressive therapeutic strategies. The goal of treatment intensification is to maximize the therapeutic benefit while carefully monitoring and managing any potential increase in adverse effects or toxicity.

The term “treatment plan”, as used herein, refers to a comprehensive and individualized strategy developed by healthcare professionals to manage a patient's medical condition. This plan outlines the specific therapeutic approaches, including medications, therapies, lifestyle modifications, and other interventions, that are recommended to achieve desired health outcomes. A treatment plan is tailored to the patient's unique needs, taking into account their medical history, current health status, and personal preferences. It serves as a roadmap for both the patient and healthcare providers, detailing the goals of treatment, the timeline for achieving these goals, and the criteria for evaluating progress. The treatment plan may be adjusted over time based on the patient's response to therapy and any changes in their condition.

The term “TBST”, as used herein, refers to Tris-Buffered Saline with Tween, such as Tween 20, a buffer solution commonly used in biological research.

The term “binding agent”, as used herein, refers to any molecule or molecular complex that has the capability to specifically recognize and bind to a target molecule. This includes, but is not limited to, antibodies, antibody fragments, antibody-like proteins, aptamers, peptides, and small molecules that can bind to antigens, receptors, or other specific targets.

The term “antigen-binding peptide”, as used herein, refers to a peptide that specifically recognizes and binds to an antigen. These antigen-binding peptides can be engineered or naturally occurring and may be used in various applications, including therapeutic agents, diagnostic assays, and research tools to detect or neutralize specific antigens. The term comprises, antibodies, antibody fragments, Fab fragments, substantially intact antibodies, chimeric antibodies, bispecific antibodies, F(ab′)2 fragments, single chain Fv fragments, single-domain antibodies as well as antibody-like proteins, such as designed ankyrin repeat proteins (DARPins), affibodies, and other engineered binding proteins.

The term “antibody”, as used herein, is used interchangeably with the term “immunoglobulin”, and refers to a glycoprotein comprising at least two heavy (H) chains and two light (L) chains inter-connected by disulfide bonds. The term also includes all recombinant forms of antibodies, e.g. antibodies expressed in prokaryotes, unglycosylated antibodies and derivatives as described below. There are five different types of heavy chains, which define antibody isotypes of different functional activity: IgM, IgD, IgG, IgA and IgE. Each heavy chain comprises a heavy chain variable region (abbreviated herein as V_{H} region) and a heavy chain constant region. Each light chain comprises a light chain variable region (abbreviated herein as V_{L} region) and a light chain constant region. The V_{H} and V_{L} regions can be further subdivided into regions of hypervariability, termed complementarity determining regions (CDR), interspersed with regions that are more conserved, termed framework regions (FR). The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “primary antibodies”, as used herein, refers to antibodies that are specifically designed to bind directly to the antigen of interest. These antibodies are used to detect and quantify the presence of specific proteins or other molecules within a sample.

The term “secondary antibodies”, as used herein, refers to antibodies that bind to primary antibodies. Secondary antibodies are typically conjugated to reporter molecules, such as enzymes, fluorophores, or radioisotopes, to facilitate the detection, quantification, or localization of the primary antibody-antigen complex. The term “secondary antibodies conjugated to reporter enzymes”, as used herein, refers to secondary antibodies that are chemically linked to enzymes capable of producing a detectable signal. Detection of said reporter enzymes belongs to the knowledge of a person skilled in the art and includes methods such as colorimetric, chemiluminescent, or fluorescent assays.

The term “antigen retrieval”, as used herein, refers to a set of techniques used in immunohistochemistry (IHC) to unmask or expose antigenic sites in formalin-fixed, paraffin-embedded tissue sections. This process enhances the binding of primary antibodies to their target antigens, which may be masked by cross-linking during the fixation process.

The term “antigen retrieval solution”, as used herein, refers to a solution used in the process of antigen retrieval in immunohistochemistry (IHC). These solutions typically contain buffers or chemicals, such as citrate buffer, EDTA, or Tris-EDTA, which help to break the cross-links formed during tissue fixation, thereby exposing the antigenic sites for antibody binding.

The terms “binds” and “binding”, as used herein, preferably relate to specific binding. In some embodiments, the term “binds” is to be understood as “is capable of binding”, and “binding” is to be understood as “capable of binding”; accordingly, the term “is specific for” is to be understood as “is capable of specifically binding (to)”, the term “specifically binds” is to be understood as “is capable of specifically binding (to)”, and “specific binding” is to be understood as “capability of specific binding (to)”.

The terms “is specific for”, “specifically binds” and “specific binding”, as used for example in the context of a molecule A being specific for a molecule B or a molecule A specifically binding to a molecule B or a molecule A showing specific binding for a molecule B, refer to a situation where molecule A binds to molecule B, but does not bind to other unrelated molecules, or with substantially reduced affinities. Such binding can be measured by routine methods, for example by competition ELISA or by measurement of affinity (K_{D}) by surface plasmon resonance measurements. Similarly, a molecule A being specific for an epitope C or a molecule A specifically binding to epitope C or a molecule A showing specific binding for epitope C, refer to a situation where molecule A binds to epitope C, but does not bind to other unrelated epitopes, or with substantially reduced affinities.

The term “H-score system”, as used herein, refers to a semi-quantitative method used to evaluate the staining intensity and distribution of immunohistochemical markers in tissue sections. The H-score is calculated by assessing the percentage of cells stained at different intensity levels (e.g., 0, 1+, 2+, 3+) and then applying a formula to generate a score that reflects both the extent and intensity of staining. The H-score is calculated using the percentage (P) of cells (0% to 100%) within a target region, where P_{O} is the proportion of negative (O) cells, P₁₊ is the percentage of 1+ cells (weak staining), P₂₊ is the percentage of 2+ cells (moderate staining), and P₃₊ is the percentage of 3+ cells (strong staining), which are summed. The H-score can be calculated using the following formula:

$$H - score = ( 0 \times {P}_{0} ) + ( 1 \times {P}_{1 +} ) + ( 2 \times {P}_{2 +} ) + ( 3 \times {P}_{3 +} )$$

This scoring system provides a numerical value that can be used to compare the expression levels of specific antigens across different samples or experimental conditions. For the expression of a biomolecule 100 is for low/normal expression while 300 is the maximum. For dichotomizing the data, a cutoff value may be used. The skilled person will understand how to choose the cutoff value based on the biomolecule and the biological sample.
For example, the H-score system may be used to score CD24 expression in cells, such as cancer cells, with 100 representing low/normal expression and 300 representing the maximum score. The expression level of CD24 in normal cells serves as the baseline, with cells showing similar intensity (+1) and receiving a score of 100. The H-score is a useful tool in case of expression heterogeneity. For dichotomizing the data, a cutoff value such as 140 may be used.

In preferred embodiments, the binding agent or antigen-binding peptide that specifically binds to CD24 is the anti-CD24 monoclonal antibody clone: ML5, also referred to as “ML5 anti-CD24 antibody”. The ML5 anti-CD24 antibody is an an IgG1 antibody, that recognizes the Leucine-Alanine-Proline protein core of CD24.

In a preferred embodiment, the ML5 anti-CD24 antibody is used with a dilution factor (antibody: buffer) in a range of about 1:10 to about 1:1000, preferably in a range of about 1:25 to 1:500, more preferably in a range of about 1:50 to about 1:250, even more preferably with a dilution factor of about 1:100; wherein, optionally, the buffer contains 1% BSA.

In one embodiment, the binding agent or antigen-binding peptide that specifically binds to CD3 is selected from the group comprising: anti-CD3 monoclonal antibody 2GV6, anti-CD3 monoclonal antibody F7.2.38, anti-CD3 monoclonal antibody LN10, or anti-CD3 monoclonal antibody SP7, preferably anti-CD3 monoclonal antibody 2GV6, or polyclonal anti-CD3 antibodies.

In one embodiment, the method according to the present invention may be used for the identification of a subpopulation of cancer, that is correlated with short survival and/or who needs a different therapeutic; wherein, preferably the cancer is nasopharyngeal cancer, even more preferably locally advanced nasopharyngeal cancer.

### BRIEF DESCRIPTION OF THE FIGURES

The present invention is now further described by reference to the following figures.

All methods mentioned in the figure descriptions below were carried out as described in detail in the examples.

FIGS. 1A-1C. ML5 anti-CD24 is verified using positive and negative controls.

(1A) Representative images of formalin-fixed paraffin-embedded (FFPE) sections of breast cancer cell lines that are (bottom) positive for CD24 and negative for CD44 (MCF-7 and SK-BR-3) or (top) negative for CD24 and positive for CD44 (MDA-MB-231 and Hs578T). Images were examined under a light microscope at 400× magnification.

(1B) Flow cytometric results for CD44/CD24 staining using the same controls as in A for comparison between the techniques.

(1C) Representative images (400×) of CD24 immunohistochemistry using M1/69 antibody in a formalin-fixed paraffin-embedded (FFPE) tissue sections of MCF-7 and SK-BR-3 cells (positive controls) and MDA-MB-231 (negative control). Sections were counterstained with hematoxylin.

FIG. 2. Specificity verification of Immunohistochemistry using anti-CD24 ML5 antibody.

Representative images (400λ) of CD24 immunohistochemistry using ML5 antibody in a formalin-fixed paraffin-embedded (FFPE) tissue section of SKBR3 cells after CD24 knockdown using specific siRNA (Si-CD24) compared with scrambled control siRNA (Si-Neg) for verification of antibody specificity. Sections were counterstained with hematoxylin.

FIGS. 3A-3B. Expression of CD24 in breast tissue.

(3A) Expression of CD24 is heterogeneous in breast cancer, with varying staining intensities. Representative images show high (+3), moderate (+2), and low (+1) staining intensities of CD24. (3B) CD24 is expressed in luminal epithelial cells of normal ducts/lobules and overexpressed in ductal hyperplasia. Representative images of formalin-fixed paraffin-embedded (FFPE) sections of normal ducts/lobules immunostained for CD44 and CD24. The black arrow indicates CD24 staining in the apical side of the luminal cells, while the gray arrow indicates CD44 staining in the myoepithelial/basal cells.

The black arrow indicates CD24 staining in the apical side of the luminal cells, while the gray indicates CD44 staining in the myoepithelial/basal cells.

FIG. 4. Impact of CD24 expression on the prognosis of breast cancer patients.

Kaplan-Meier survival curve demonstrates disease-free survival (DFS) of breast cancer patients based on CD24. Statistical significance was determined using the log-rank test.

FIGS. 5A-5D. Representative Images for Immunohistochemical Sections.

Representative images for nuclear BMI1 (5A), membranous CD44 (5B), primarily cytoplasmic ALDH1, alone or with CD44 (5C) and primarily cytoplasmic CD24, alone or with CD44 (membranous) (5D). White arrows indicate cancer cells, while black arrows show immune infiltrating cells.

FIGS. 6A-6C. Prognostic ability of CSC makers in LA-NPC.

Kaplan-Meier survival curves show the disease-free survival (DFS) (6A), metastasis-free survival (MFS) (6B) or overall survival (OS) (6C) of LA-NPC patients in relation to CSC markers. Statistical significance was calculated using the log-rank test.

FIGS. 7A-7B. Expression of ALDH1 and CD24 is heterogeneous in NPC with different staining intensities.

Representative images with high (+3), moderate (+2), and low (+1) staining intensity of ALDH1 (7A) or CD24 (7B).

FIG. 8. Based on their H-Score, the prognostic ability of ALDH1 and CD24 CSC makers in LA-NPC.

Kaplan-Meier survival curves show the disease-free survival (DFS), metastasis-free survival (MFS) or overall survival (OS) of LA-NPC patients in relation to ALDH1 (bottom) or CD24 (top) CSC markers scored based on their H-Score. Statistical significance was calculated using the log-rank test.

FIGS. 9A-9B. The prognostic ability of CD3+ TIL in LA-NPC.

(9A) Representative image for High CD3+ TIL (scores 4 and 3) vs Low CD3+ TIL (scores 2 and 1). (9B) Kaplan-Meier survival curve showing the effect of CD3+ TIL on survival (DFS, MFS and OS). Statistical significance was calculated using the log-rank test.

FIGS. 10A-10B. The prognostic ability of CD3+ TIL combined with Tumor CD24 expression in LA-NPC.

Kaplan-Meier survival curves show the effect of the different combinations of CD3+ TIL and Tumor CD24 (10A) or Tumor CD24/CD44 (10B) on disease-free survival (DFS), metastasis-free survival (MFS) or Overall Survival (OS). Statistical significance was calculated using the log-rank test.

In the following, reference is made to the examples, which are given to illustrate, not to limit the present invention.

### EXAMPLES

The following Examples demonstrate how the newly identified combination of low CD3+ TIL and CD24+ cells can be reliably used as an indicator in the diagnosis, prognosis, stratification and/or monitoring of a therapy, of a cancer disease in a subject.

### Example 1

### Material and Methods

### Patients

This study was conducted under the Helsinki Declaration and approved by the Research Advisory Council of King Faisal Specialist Hospital and Research Centre (KFSH&RC). The archived (formalin-fixed paraffin-embedded) FFPE tissues were derived from a cohort of breast cancer patients diagnosed with invasive ductal carcinoma (IDC), treated between 2006-2008. All patients provided informed consent.

### Immunohistochemistry

Sections (4 microns) of FFPE breast cancer tissue blocks were dewaxed in xylene and rehydrated using alcohol/water gradients. Antigen retrieval was performed in a Decloaking Chamber pressure cooker, as detailed in Table 1. Endogenous peroxidase was quenched with 0.9% H₂O₂, and endogenous biotin was blocked using biotin and avidin with 3 washes in between. Primary antibodies, diluted in 1% bovine serum albumin, were incubated overnight at 4° C. The two-species double staining method was employed, with CD44 as a rabbit antibody and CD24 as a mouse antibody. Biotinylated anti-mouse secondary antibody was applied first, followed by Streptavidin Alkaline Phosphatase (both are ready to use), with washing steps in between. Envision anti-rabbit horseradish peroxidase was then used, followed by Fast Red (30 minutes) and 3,3′-Diaminobenzidine (10 minutes). All washes were performed using Tris-buffered saline with Tween.

Scoring was limited to IDC cells only, disregarding expression in tumor-associated immune cells, normal ducts/lobules, and ductal carcinoma in situ. The expression of nuclear CD24 was not considered in this study.

Due to expression heterogeneity, the H-score system was utilized to score CD24 expression in cancer cells, with 100 representing low/normal expression and 300 representing the maximum score. The expression level of CD24 in normal luminal cells served as the baseline, with cancer cells showing similar intensity (+1) and receiving a score of 100. The data were dichotomized using 140 as a cutoff. Similarly, the H-score system was used to score Ep-CAM expression in cancer, with a cutoff for Ep-CAM overexpression set at 200.

TABLE 1
Conditions used for manual IHC in FFPE tissues

| | Antigen Retrieval | | Primary | Secondary |
|---|---|---|---|---|
| Antibody | Solution | Conditions* | Antibody | antibody |
| CD24/CD44 | Tris- | 121° C./7 | CD24, | RTU** |
| | EDTA | minutes | 1:100 | ^{#} Biotinylated goat |
| | pH 9 | | CD44, | anti-mouse AP |
| | | | 1:500 | & ^{⊥} Envision anti- |
| | | | | rabbit HRP |
| Ep-CAM | Citrate | 121° C./7 | 1:200 | ^{⊥} Envision goat |
| | | minutes | | anti-mouse |

| | |
|---|---|
| Vimentin | Previously done and described |
| E-Cadherin | Previously done and described |
| SKP-2 | Previously done and described |
| p21&p27 | |

*The indicated temperature is SP1, while SP2 is always 95° C. for 10 min.
*** RTU, Ready to use
^{#} 4+ Biotinylated goat anti-mouse from Biocare Medical, Cat# GM601H followed by washing and 4+ Streptavidin-AP Obtained from Biocare Medical, Cat# AP605H
^{⊥} Obtained from Dako/Agilent

### Flow Cytometry

Briefly, the tissues were digested using a collagenase digestion medium. The cells were then cleaned using DNase, filtered through a 30-micron mesh, and stained with various antibodies, including Ep-CAM-APC, CD49f-PE, CD44 APC-Cy7, CD24 PE-Alexa 610, and CD45 Alexa 430. Additionally, 4′,6-diamidino-2-phenylindole (DAPI) was added for viability assessment.

The cells were initially gated based on forward and side scattering to identify cell-like structures. Viable cells (DAPI-negative) were then selected, gating on single cells only. Subsequently, either CD45-positive (population 5 or p5) or CD45-negative (p4) cells were selected. Further gating was performed on epithelial cells using Ep-CAM (p6) from the CD45-negative cell population.

CD24^{hi} cells (MCF-7, SK-BR-3) and CD44^{hi} cells (MDA-MB-231 and Hs578T) were also stained with CD44 APC-Cy7, CD24 PE-Alexa 610, or an isotype control.

### Results

Verification of CD24 Immunohistochemical Staining with Anti-CD24 (ML5) Using Multiple Strategies

To optimize the CD44/CD24 IHC double staining methodology in FFPE sections, various antigen retrieval solutions and temperatures were tested. The best results, characterized by a high signal and a low background, were achieved under the conditions outlined in Table 1.

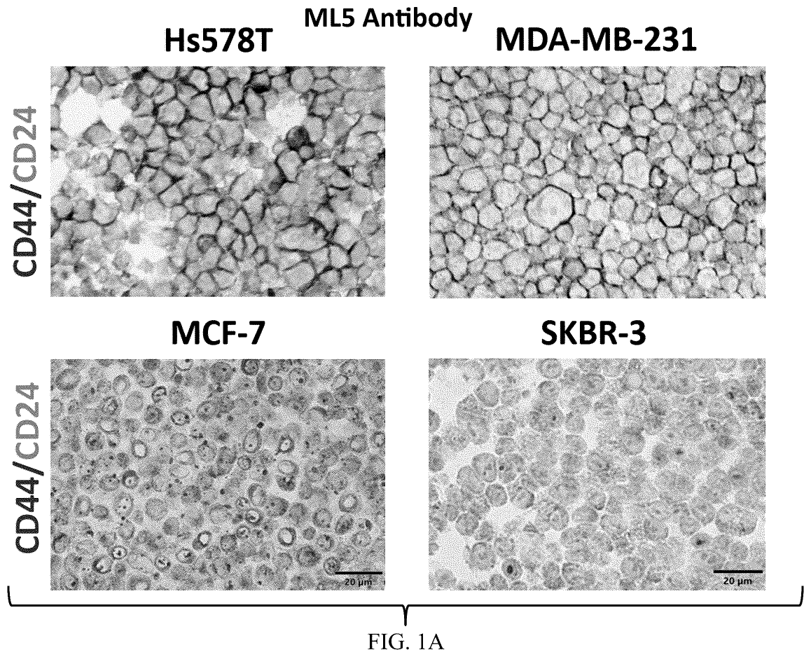

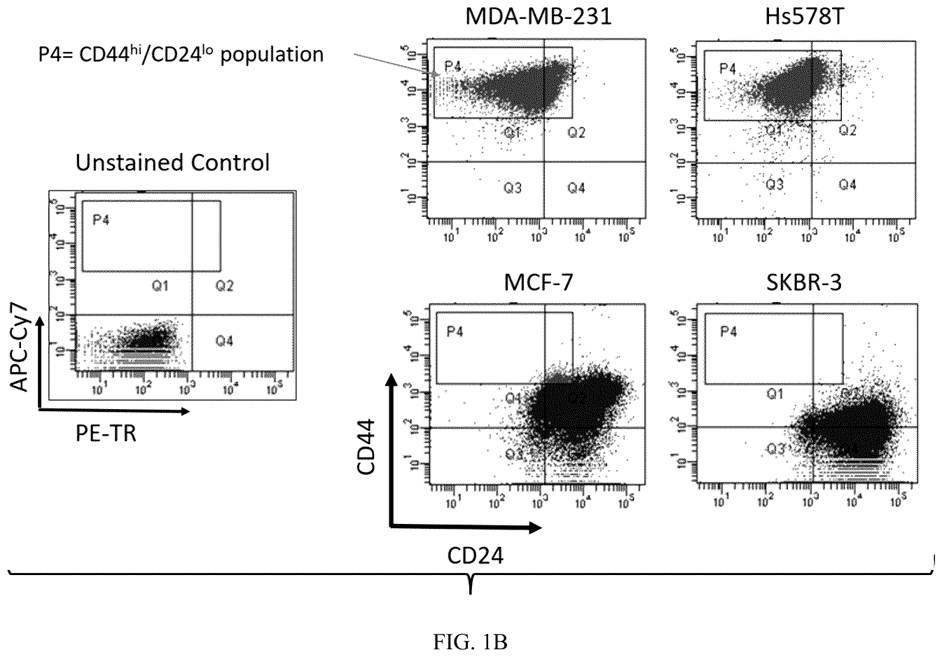

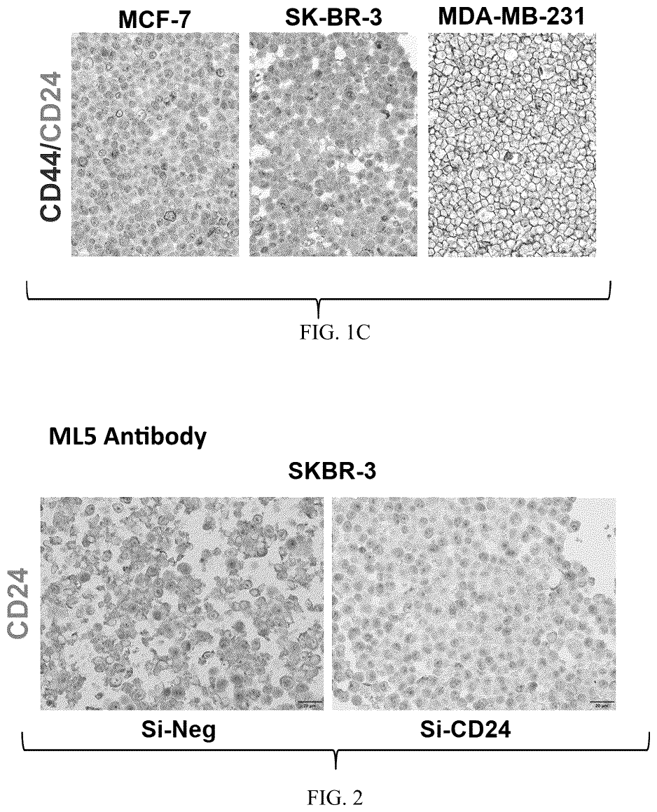

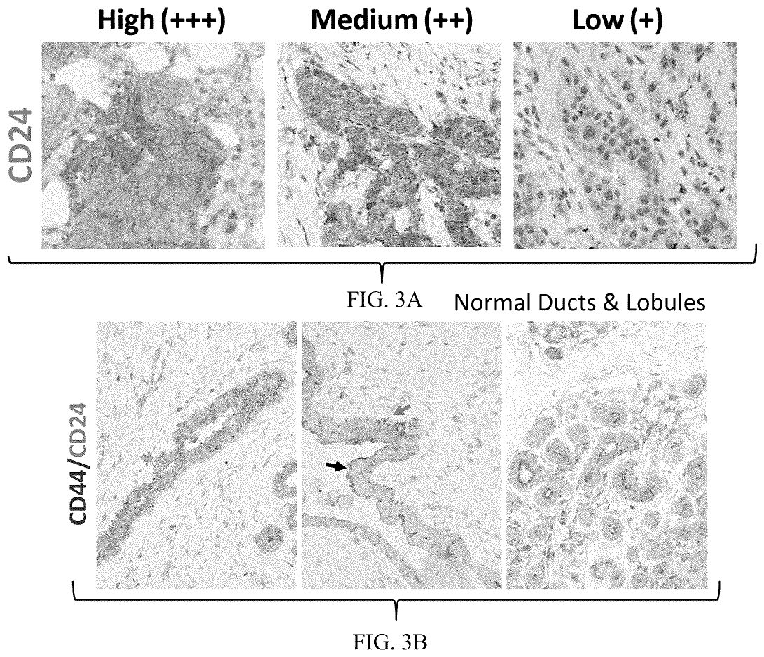

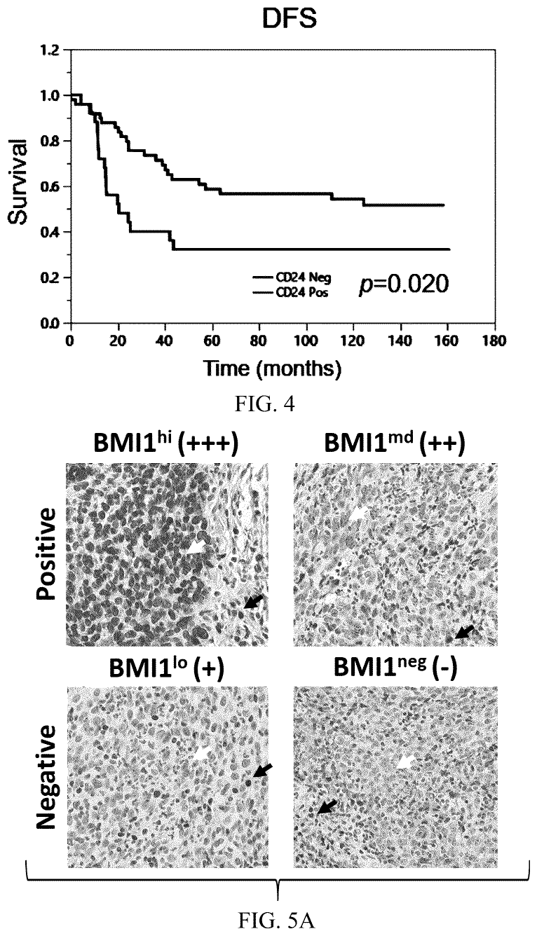

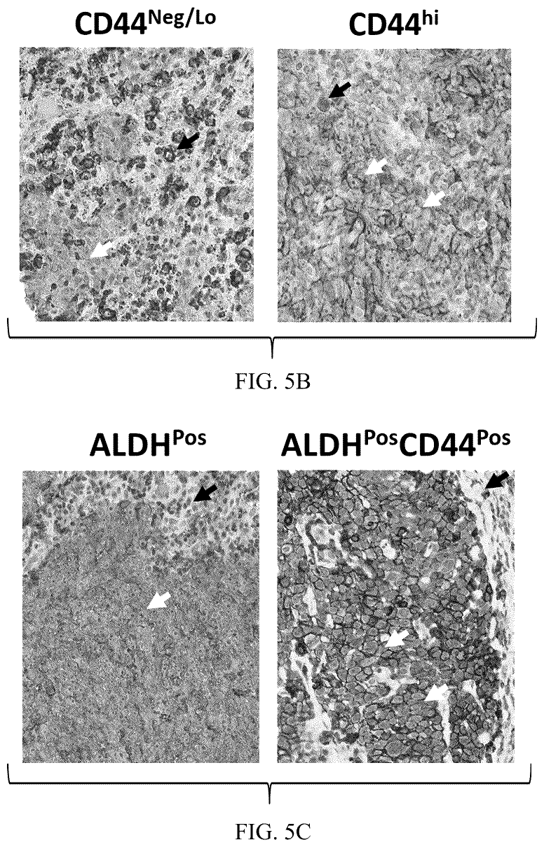

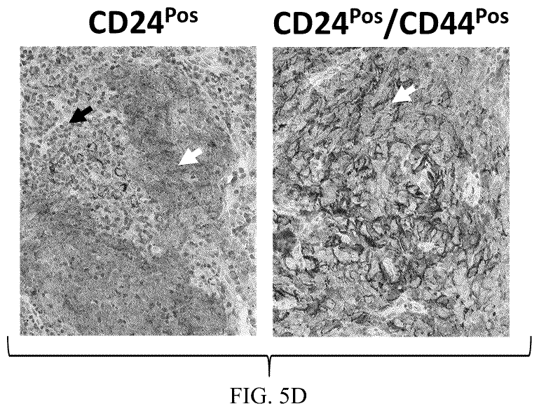

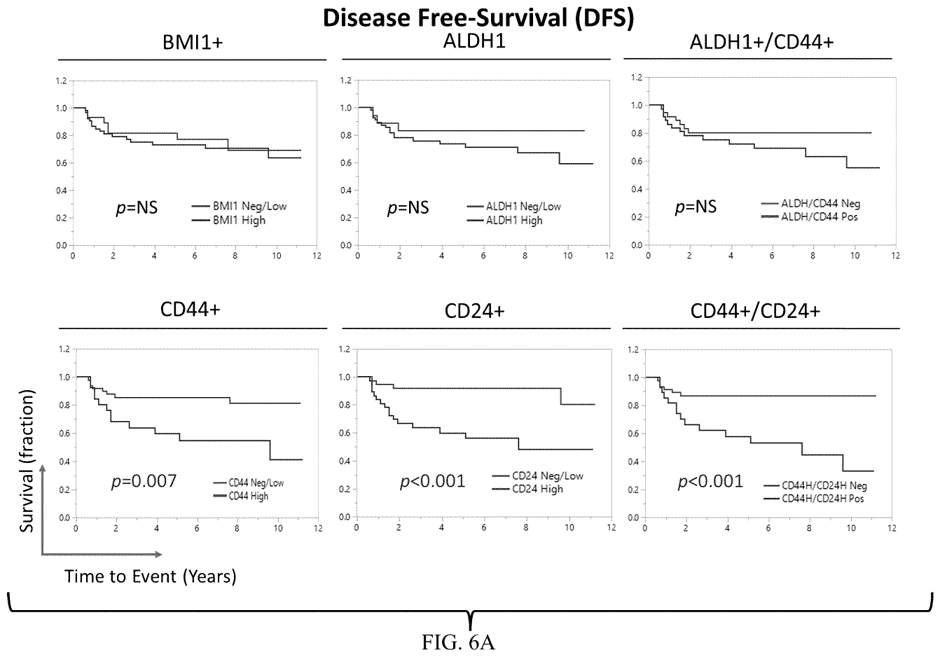

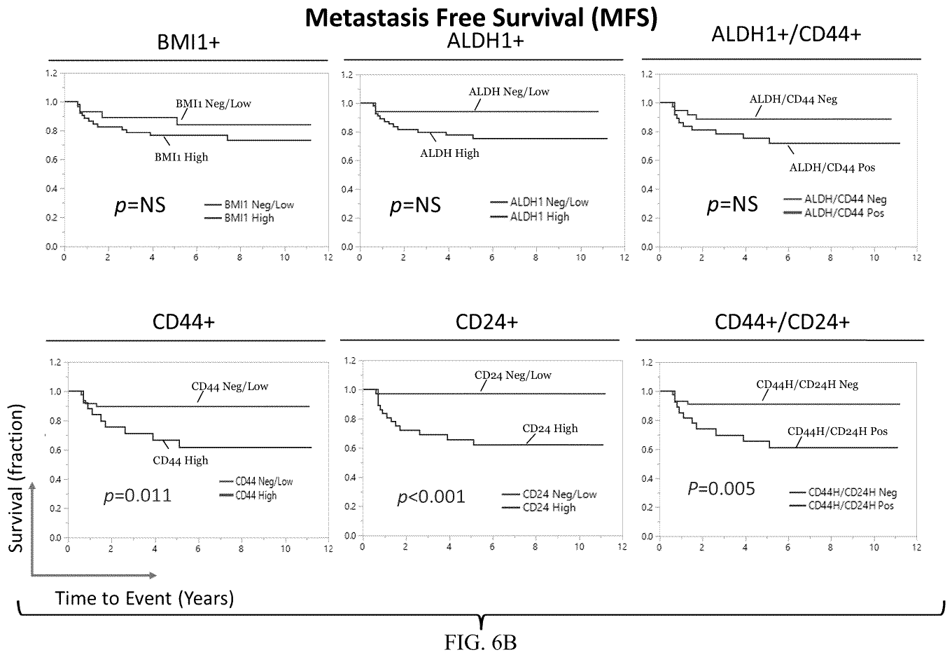

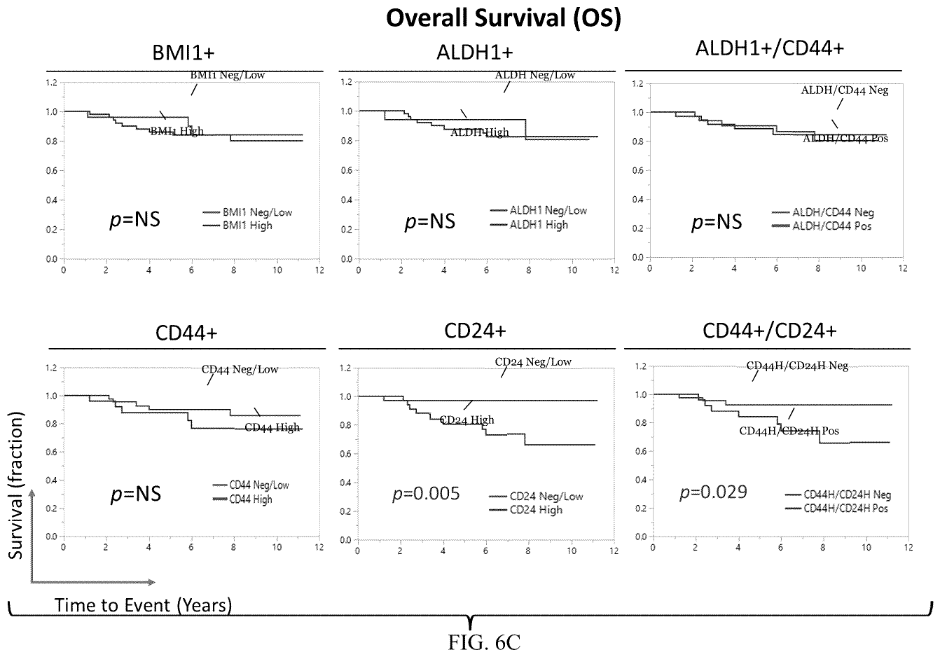

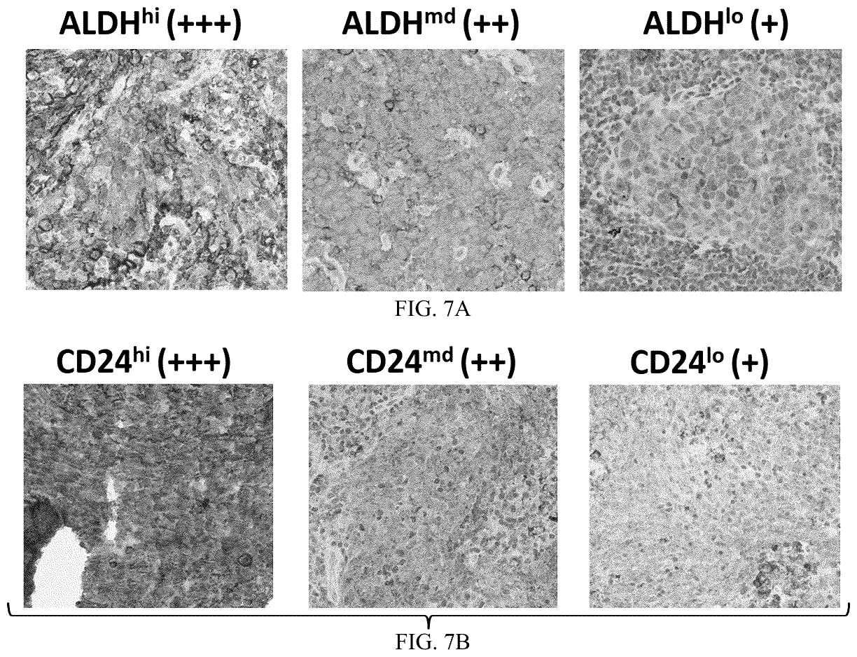

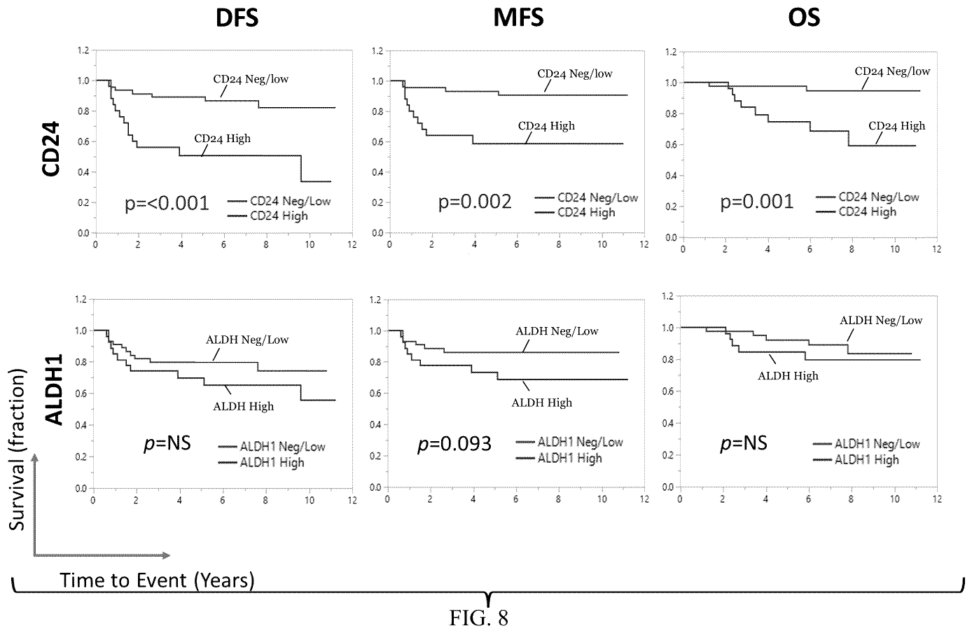

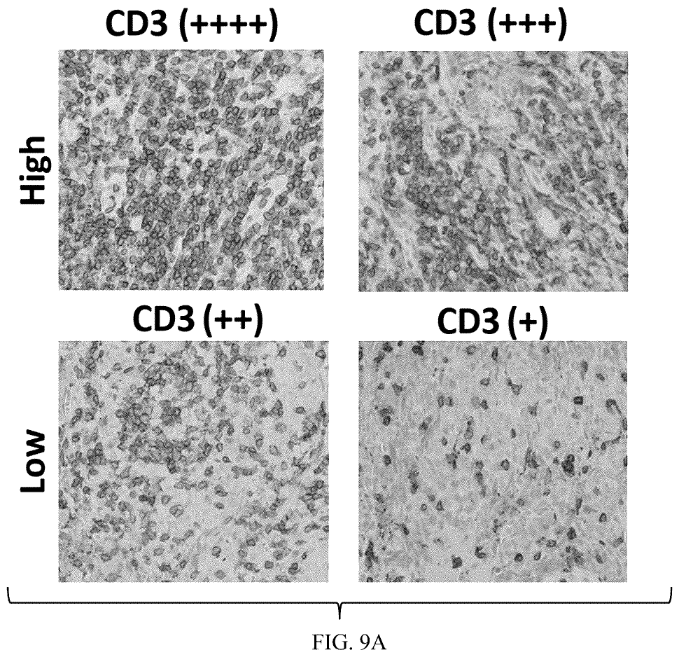

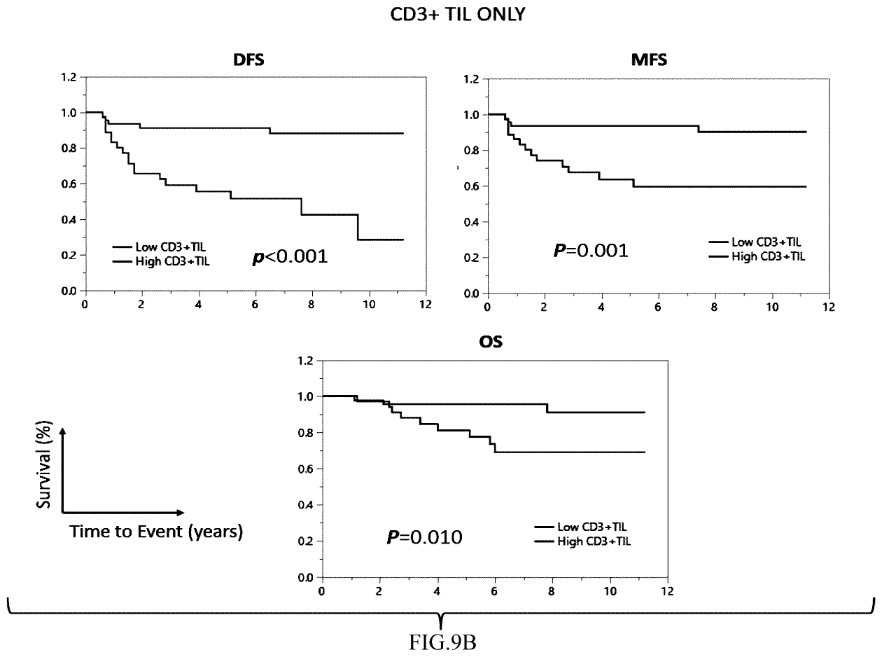

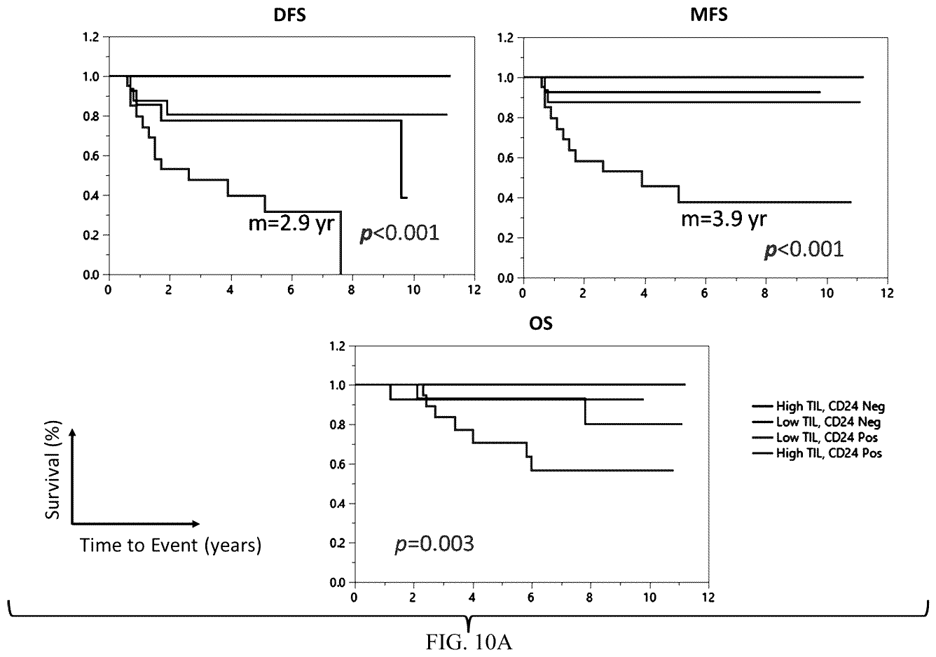

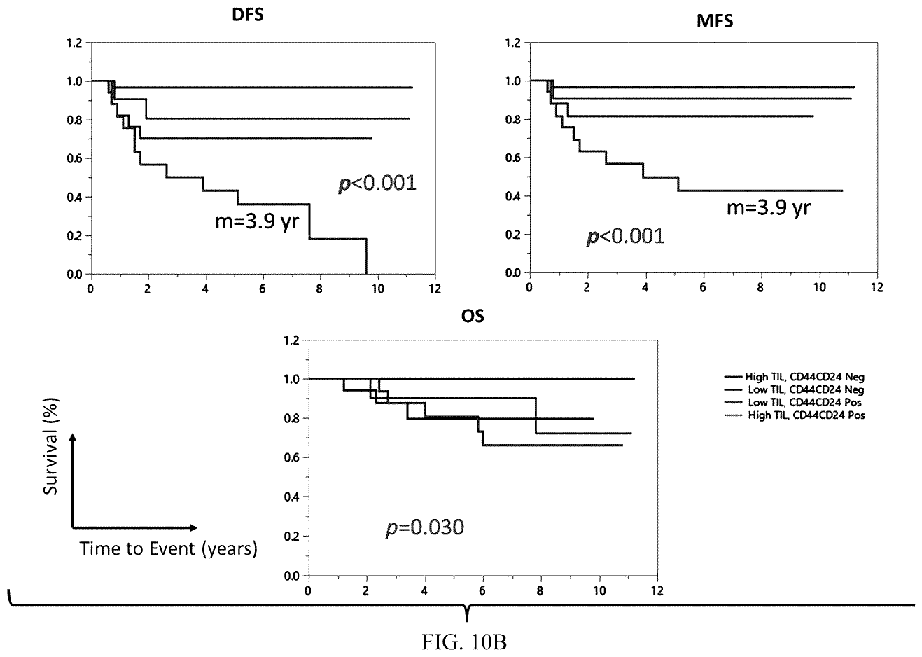

To validate CD44/CD24 double staining, positive and negative controls were utilized. The SK-BR-3 and MCF-7 breast cancer cell lines served as CD24 positive and CD44 negative controls, respectively, while the MDA-MB-231 and Hs578T breast cancer cell lines were used as CD24 negative/low and CD44 positive controls (FIG. 1A). The IHC results (FIG. 1A) aligned with flow cytometry findings, demonstrating highly positive CD44 expression in MDA-MB231 and Hs578T and low expression in MCF-7 and SK-BR-3, while CD24 expression was highly positive in MCF-7 and SK-BR-3 and low in MDA-MB-231 and Hs-578T (FIG. 1B).

In addition, two additional verification methods were employed in accordance with the recommendations of the ad hoc International Working Group for Antibody Validation. An independent antibody strategy was also employed, where staining with another anti-CD24 antibody (clone M1/69) yielded similar results to the ML5 anti-CD24 antibody in cell lines (FIG. 1C) and a genetic strategy involved CD24 knockdown using specific siRNA, resulting in the loss of detection by the ML5 anti-CD24 antibody (FIG. 2). Altogether, the genetic and independent antibody strategies validated the specificity of the ML5 anti-CD24 antibody IHC in FFPE sections.

### Example 2

All methods mentioned in this example were carried out as described in Example 1.

CD24 Overexpression Correlates with Increased Cell Proliferation and Shorter Disease-Free Survival (DFS) in Breast Cancer Tissues

In patients with breast cancer, CD24 expression was predominantly localized in the cytoplasm, with varying staining intensities ranging from mild (+) to strong (+++) (FIG. 3A). Therefore, the H-score method was used to quantify the staining intensity. The mild intensity (+) of CD24 in cancer cells was similar to that observed in luminal cells of normal ducts (FIG. 3B). CD24 overexpression was observed in 33.8% of patients and was significantly correlated with higher histological grade (p=0.013) and estrogen receptor negativity (p=0.010). Notably, CD24 overexpression was positively correlated with proliferation markers, such as Ki-67 positivity (p=0.002) and SKP2 overexpression (p=0.009), and negatively correlated with the expression of the cell cycle inhibitor p21 (p=0.047). Moreover, CD24 overexpression was found to be correlated with shorter DFS (p=0.020) (FIG. 4).

### Example 3

### Material and Methods

This study is a retrospective, single-institution cohort study testing the effect of different CSCs markers on the survival of LA-NPC patients.

This study was conducted according to the Declaration of Helsinki and the guidelines of the Institutional Review Boards (IRBs) of King Faisal Specialist Hospital and Research Centre (Research Ethics Committee, REC, and Basic Research Committee BRC), which approved the work under a RAC #2150-013. The need for consent has been waived as the study was retrospective, and patients were not individually identifiable.

### Patient Selection

NPC patients were enrolled from previous trial testing the effect of small dose irradiation during the induction neoadjuvant chemotherapy (INC) and before the concurrent chemo-radiotherapy, which ended with negative results showing no significant effect of small dose INC on patient outcome. 83 out of 108 enrolled patients had tissue blocks available for analysis (42 in the control arm and 41 in the experimental arm).

### Immunohistochemistry (IHC)

Formalin-fixed paraffin-embedded (FFPE) NPC tissue blocks obtained at the time of diagnosis were used for this study. Dewaxed and rehydrated 4 μm sections were immunostained for CD3+ and vimentin using a fully automated Ventana Benchmark Ultra system as previously described or stained manually for BMI1, CD24, ALDH1 and CD44.

BMI1 was stained using primary anti-mouse BMI1 (clone F6, diluted 1:300), followed by the Envision® anti-mouse secondary antibody.

### CD24/CD44 and ALDH1/CD44 Doublestaining

Sections were blocked for Biotin/Avidin, followed by protein block using 10% goat serum. Primary antibodies, used overnight at 4° C., were mouse anti-CD24 antibody (clone ML5) or mouse anti-ALDH1 antibody (clone 44/ALDH, BD Biosciences, diluted 1:200), mixed with rabbit anti-CD44 antibody (Cat #HPA005785, Sigma, St. Louis, USA, diluted 1:500). Secondary antibodies were 4+ Goat anti-mouse IgG, was used, followed by 4+ Streptavidin Alkaline phosphatase (both from Biocare medical, Cat #GM601H and AP605H respectively), followed by the Envision anti-rabbit secondary antibody. The signals were visualized using a sequential approach, with Fast Red being first, followed by DAB.

### Pathological Scoring

CD3+ TIL were scored based on the percentage of area, CD3+ TIL were occupying from the tumor section. Score 1 was used when CD3+ TIL occupy from 0 up to 10% of the tumor section, score 2 defined as CD3+ TIL occupying more than 10% but up to 40% of the tumor (Both Score 1 and 2 are considered low CD3+ TIL). Score 3 was used for CD3+ TIL occupying >40 and ≤70% of tumor area and Score 4 for CD3+ TIL occupying more than 70% of tumor area. (High CD3+ TIL=Score 3 or 4). BMI1 intensity was given an intensity score of 0-3, where 0 is negative, 1 is mild, 2 is moderate, and 3 is intense staining. BMI1 staining intensity results were further dichotomized where scores 0 and 1 were considered negative, and scores 2 and 3 were considered positive. ALDH1 and CD24 were scored using a 5-10% increment, with 10% and 30% as a cutoff for ALDH1 and CD24, respectively. ALDH1 and CD24 staining was heterogeneous; thus, the H-score was also used in a different analysis, as previously reported, using the score of 90 as the cutoff. Membranous CD44 was scored using a 5-10% increment with ≥70% as a cutoff. Similarly, ALDH1/CD44 was scored using a 5-10% increment with 10% as a cutoff. Similarly, CD24/CD44 double-positive cancer cells were scored using a 5-10% increment with 10% as a cutoff.

### Statistical Analysis

The correlation between CSCs markers and survival outcomes was evaluated using the Cox proportional hazard model. Survival plots were generated using the Kaplan-Meier method and the curves were compared using the log-rank test. Time was censored for patients alive or disease-free at the last follow-up. Categorical variables were compared using the Fisher Exact test. The p-value of 0.05 was the threshold for significance.

### Example 4

All methods mentioned in this example were carried out as described in Example 3.

### Patient Characteristics

The majority of patients were males with large tumors (T3 and T4) and advanced lymph node involvement (N), which is consistent with LA-NPC (Table 2). The tumors were predominantly non-keratinizing undifferentiated carcinoma, i.e., WHO type III histological subtype. LA-NPC patients were followed up for a median time of 8 years from the time of diagnosis, of which 23 (28%) relapsed, including 6 patients with local or loco-regional and another 17 (20%) with systemic relapse. Of all the LA-NPC patients, eventually, 12 (14%) died from the disease.

TABLE 2
Patient Characteristics

| Features | Categories | # Patients | |
|---|---|---|---|
| Age | | | |
| Range 18-72 | <40 years | 32 | *(39) |
| Median 45 | ≥40 years | 51 | (61) |
| Gender | Male | 62 | (75) |
| | Female | 21 | (25) |
| WHO Type | I | 0 | (0) |
| | II | 5 | (6) |
| | III | 78 | (94) |
| T stage | T1 | 26 | (31) |
| | T2 | 2 | (2) |
| | T3 | 30 | (36) |
| | T4 | 25 | (30) |
| N stage | N0 | 5 | (6) |
| | N1 | 12 | (15) |
| | N2 | 24 | (29) |
| | N3 | 42 | (51) |
| TNM Staging | III | 24 | (29) |
| | IV | 59 | (71) |
| Relapse | No | 60 | (72) |
| | Yes | 23 | (28) |
| Type of | None | 60 | (72) |
| Relapse | Local | 3 | (4) |
| | Locoregional | 3 | (4) |
| | Systemic | 17 | (20) |
| Survival | Alive | 71 | (86) |
| | Dead | 12 | (14) |

*Percentage of cases

Among NPC CSC Markers, CD24 Expression Correlated Significantly with Survival.

BMI1 was nuclear, and its intensity in tumor cells was variable between “Low,” i.e., negative/low (+) intensity versus “high,” i.e., moderate (++)/high (+++) intensity (white arrows), (FIG. 5). In contrast, the intensity of BMI1 in immune cells was more or less consistent and thus was used as an internal control (black arrow). There was high BMI1 expression in 65% of cases, which did not correlate with DFS, MFS or OS (FIGS. 6A-6C).

CD44 was membranous, and it was overexpressed in cancer cells in 34% of cases. In addition, it was overexpressed in immune infiltrating cells. High tumor CD44 expression (>70% of cells) correlated significantly with DFS (p=0.007) and MFS (p=0.01). The correlation of CD44 expression with OS was not significant.

ALDH1 was mostly cytoplasmic and overexpressed in tumor cells in 76% of cases (FIG. 5). In addition, it was expressed by some histiocytes/macrophages (data not shown). There was no significant correlation between ALDH1 expression in tumor cells and patient survival (FIG. 6A-6C). Some cases had cancer cells that were positive for both ALDH1 and CD44 (ALDH1/CD44). Cancer cells with combined ALDH1 and CD44 expression were present in 50% of NPC cases. There was no correlation between ALDH1/CD44 expression and survival.

ALDH1 staining intensity was heterogeneous (FIG. 7A). Therefore, ALDH1 was further scored using H-score. ALDH1 staining scored by H-score did not correlate with DFS, MFS or OS (FIG. 8).

CD24 was overexpressed in 50% of NPC cases. There was a significant correlation between CD24 and DFS (p<0.001), MFS (p<0.001), and OS (p=0.005) (FIGS. 6A-6C). Similarly, NPC cases with combined CD24 and CD44 expression represented 38% of NPC cases, and this CD24/CD44 expression correlated significantly with DFS (p<0.001), MFS (p=0.005) and OS (p=0.029).

CD24 staining intensity was also heterogeneous (FIG. 7B). Therefore, CD24 was scored further using the H-score. CD24 staining scored by H-score correlated significantly with DFS (p<0.001), MFS (p=0.002) and OS (p=0.001) (FIG. 8).

Altogether, among tested CSCs markers, CD24 overexpression, alone or combined with CD44, whether scored in 5-10% increments or using the H-score, correlated significantly with shorter DFS, MFS and OS.

### Example 5

All methods mentioned in this example were carried out as described in Example 3.

Expression of the EMT Marker, Vimentin, Correlates with DFS.

Epithelial-to-mesenchymal transition (EMT) is associated with the generation of CSC. The prognostic ability of vimentin in LA-NPC was analyzed. Vimentin was expressed in 31% of NPC cases (data not shown). In addition, Vimentin was heavily expressed in immune infiltrating cells (data not shown). Vimentin expression in cancer cells correlated significantly with DFS, while the correlation with MFS and OS did not reach significance (data not shown).

Low CD3+ TIL Correlated with Shorter Survival.

The prognostic ability of the immune prognostic marker CD3+ TIL was tested. Low CD3+ TIL (CD3+ TIL scores 1 and 2) vs High CD3+ TIL (CD3+ TIL scores 3 and 4) (FIG. 9A) correlated significantly with DFS (p<0.001), MFS (p=0.001) and OS (p=0.010) (FIG. 9B).

### Example 6

CD24 expression is an independent prognostic marker in LA-NPC Cox regression analysis was used to examine whether the tested CSC markers, including CD24, were dependent on other factors. Initially, univariate Cox regression analysis was applied to each CSC marker and Vimentin. Among tested CSC makers, CD44, CD24, and the CD44/CD24 combination correlated significantly with DFS (Table 3), while only CD24 and CD44/CD24 correlated significantly with OS. Vimentin correlated significantly with DFS (p=0.021) but not OS. As demonstrated previously, low CD3+ TIL correlated significantly with shorter DFS and OS. As indicated previously, the trial arm (with different aim not related to this study) had no significant effect on the prognosis of NPC patients.

Multivariate Cox regression analysis was performed to determine independent factors that correlate with the survival of LA-NPC in the patients. In multivariate analysis, only CD3+ TIL and CD24 correlated significantly with DFS (Table 4). CD24 correlated significantly with OS, while CD3+ TIL was borderline. Altogether, CD24 expression and CD3+ TIL were the only independent prognostic factors that correlated significantly with survival in multivariate analysis.

TABLE 3
Univariate Cox proportional hazard regression analysis of the different clinicopathological features
and biomarkers with disease-free survival (DFS) and Overall Survival (OS) in 83 patients with LA-NPC.

| | Relapse | | DFS | | | Death | | OS | | |
|---|---|---|---|---|---|---|---|---|---|---|
| | − | + | HR | 95% CI | *p | − | + | HR | 95% CI | *p |
| BMI1** | | | | | | | | | | |
| Neg | 22 (76) | 7 (24) | 1 | | | 26 (90) | 3 (10) | 1 | | |
| Pos | 37 (70) | 16 (30) | 1.2 | 0.5-2.9 | 0.700 | 44 (83) | 9 (17) | 1.4 | 0.4-5.3 | 0.596 |
| CD44 | | | | | | | | | | |
| Neg (<70%) | 41 (84) | 8 (16) | 1 | | | 44 (90) | 5 (10) | 1 | | |
| Pos (≥70%) | 13 (52) | 12 (48) | 3.2 | 1.3-7.9 | 0.011 | 20 (80) | 5 (20) | 1.9 | 0.6-6.7 | 0.298 |
| ALDH1^{⋄} | | | | | | | | | | |
| Neg (<10%) | 15 (83) | 3 (17) | 1 | | | 33 (87) | 5 (13) | 1 | | |
| Pos (≥10%) | 39 (70) | 17 (30) | 2.0 | 0.6-7.0 | 0.256 | 31 (86) | 5 (14) | 1.4 | 0.3-6.7 | 0.659 |
| ALDH1/CD44^{⋄} | | | | | | | | | | |
| Neg (<10%) | 30 (81) | 7 (19) | 1 | | | 37 (86) | 6 (14) | 1 | | |
| Pos (≥10%) | 24 (65) | 13 (35) | 1.8 | 0.7-4.5 | 0.211 | 27 (87) | 4 (13) | 0.9 | 0.3-3.2 | 0.932 |
| CD24^{⋄} | | | | | | | | | | |
| Neg (<30%) | 33 (89) | 4 (11) | 1 | | | 43 (93) | 3 (7) | 1 | | |
| Pos (≥30%) | 21 (57) | 16 (43) | 5.3 | 1.8-16.0 | 0.003 | 21 (75) | 7 (25) | 10.8 | 1.4-85.5 | 0.024 |
| CD24/CD44^{⋄} | | | | | | | | | | |
| Neg (<10%) | 40 (87) | 6 (13) | 1 | | | 43 (93) | 3 (7) | 1 | | |
| Pos (≥10%) | 14 (50) | 14 (50) | 4.6 | 1.7-11.9 | 0.002 | 21 (75) | 7 (25) | 4.0 | 1.0-15.6 | 0.044 |
| Vimentin | | | | | | | | | | |
| Neg | 46 (81) [symbol] | 11 (19) | 1 | | | 51 (89) | 6 (11) | 1 | | |
| Pos | 14 (54) | 12 (46) | 2.6 | 1.2-6.0 | 0.021 | 20 (77) | 6 (23) | 2.4 | 0.8-7.4 | 0.134 |
| CD3 + TIL | | | | | | | | | | |
| High | 42 (89) | 5 (11) | 1 | | | 44 (94) | 3 (6) | 1 | | |
| Low | 18 (50) | 18 (50) | 6.5 | 2.4-17.8 | <0.001 | 27 (75) | 9 (25) | 4.8 | 1.3-17.7 | 0.020 |
| Trial Arm [symbol] | | | | | | | | | | |
| Control arm | 32 (76) | 10 (24) | 1 | | | 35 (83) | 7 (17) | 1 | | |
| LDXRT | 28 (68) | 13 (32) | 1.5 | 0.7-3.5 | 0.313 | 36 (88) | 5 (12) | 1.27 | 0.4-4.0 | 0.688 |

Abbreviations: (+ and −) are numbers of patients,
*p values in bold represent significant data, [symbol]
Numbers between brackets are the percentages of patients,
**1 sample were missing for BMI1,
^{⋄}9 samples were missing for ALDH1 and CD24. [symbol]
Control arm = No irradiation during neoadjuvant chemotherapy, LDXRT = Lose dose of irradiation during the neoadjuvant chemotherapy.

TABLE 4
Multivariate Cox proportional hazard regression analysis of the different CSCs markers
with disease-free survival (DFS) and overall survival (OS) in 83 patients with LA-NPC.

| | Relapse | | DFS | | | Death | | OS | | |
|---|---|---|---|---|---|---|---|---|---|---|
| | − | + | HR | 95% CI | *p | − | + | HR | 95% CI | *p |
| WHO Type | | | | | | | | | | |
| III | 59 (76) [symbol] | 19 (24) | 1 | | | 67 (86) | 11 (14) | 1 | | |
| I & II | 1 (20) | 4 (80) | 3.2 | 0.9-11.7 | 0.080 | 4 (80) | 1 (20) | 2.1 | 0.2-21.8 | 0.541 |
| Vimentin | | | | | | | | | | |
| Neg | 46 (81) | 11 (19) | 1 | | | 51 (89) | 6 (11) | 1 | | |
| Pos | 14 (54) | 12 (46) | 1.1 | 0.4-3.2 | 0.819 | 20 (77) | 6 (23) | 1.3 | 0.3-5.3 | 0.714 |
| CD44^{⋄} | | | | | | | | | | |
| <70% | 41 (84) | 8 (16) | 1 | | | 44 (90) | 5 (10) | 1 | | |
| ≥70% | 13 (52) | 12 (48) | 2.0 | 0.7-5.1 | 0.178 | 20 (80) | 5 (20) | 1.0 | 0.2-3.7 | 0.952 |
| CD24^{⋄} | | | | | | | | | | |
| <30% | 33 (89) | 4 (11) | 1 | | | 36 (97) | 1 (3) | 1 | | |
| ≥30% | 21 (57) | 16 (43) | 4.3 | 1.3-13.9 | 0.015 | 28 (76) | 9 (24) | 8.9 | 1.1-73.0 | 0.041 |
| CD3 + TIL | | | | | | | | | | |
| High | 42 (89) | 5 (11) | 1 | | | 44 (94) | 3 (6) | 1 | | |
| Low | 18 (50) | 18 (50) | 7.4 | 1.9-28.6 | 0.004 | 27 (75) | 9 (25) | 4.9 | 0.9-26.9 | 0.068 |

Abbreviations: (+ and −) are numbers of patients, Neg = Negative, Pos = Positive,
*p values in bold and shaded represent significant data, [symbol]
Numbers between brackets are the percentages of patients,
^{⋄}9 samples were missing for CD24.

As CD3+ TIL and CD24 tumor expression were the only two independent prognostic factors that correlated with DFS in the multivariate analysis, these factors were combined and their combined correlation was tested. Low CD3+ TIL with high tumor CD24 expression emerged as a subgroup of LA-NPC with the shortest DFS and MFS, and OS (FIG. 10A). The same was true for low CD3+ TIL with CD44/CD24 i.e. patients with Low CD3+ TIL and high CD44/CD24 tumor expression had the worst DFS and MFS, and OS (FIG. 10B).

### Example 7

In summary the Examples of the present application convincingly show that the combination of low CD3+ TIL and CD24+ expression cells is a reliable prognostic factor in cancer diseases.

IHC is considered a standard technique for studying tumor tissues, as it preserves the in vivo tissue architecture and avoids artifacts generated sometimes in cell culture. However, this technique is heavily dependent on well-validated antibodies for accurate results. Despite the availability of well-characterized antibodies for selected biomarkers, such as anti-CD44 antibodies, identifying reliable anti-CD24 antibodies specifically suited for IHC has so far remained challenging in the prior art.

In this application, the present inventors optimized a methodology to detect CD24 expression in IHC using anti-CD24 antibodies (ML5 and M1/69 clones). The specificity of these antibodies was rigorously verified in FFPE IHC using multiple validation approaches set by the ad hoc International Working Group for Antibody Validation guidelines. Given the scarcity of well-validated anti-CD24 antibodies for FFPE IHC and the significance of CD24 in tumor biology, this application provides a valuable tool to study CD24 in archived clinical samples.

Nasopharyngeal Carcinoma responds very well to concurrent chemo-radiotherapy (CCRT). However, a subset of patients relapse and have shorter survival. In specific geographical locations, including Saudi Arabia, the majority of patients present with locally advanced nasopharyngeal carcinoma. By definition, all patients are TNM stage 3 or 4A of the AJCC classification, and thus, prognostication using TNM staging is limited. In the present application multiple CSC makers were screened side-by-side as prognostic factors in LA-NPC. The expression of CD24 in >30% of tumor cells correlated significantly with disease-free survival, metastasis-free survival, and overall survival. Unexpectedly, the present application demonstrates for the first time that the cancer stem cell (CSC) marker CD24, in combination with CD3+ TIL, is significantly prognostic. Patients with LA-NPC tumors with low CD3+ TIL while enriched with CD24+ CSC-like cells have significantly shorter DFS (median 2.9 years), MFS (median 3.9 years) and OS.

By this, this study breaks new ground in cancer prognosis by merging immune-related and cancer stem cell factors. The results of the present examples demonstrate for the first time that the analyzing the combination of the presence of CD24 positive cells and the level of CD3+ TIL is highly suitable to be used in a method for the diagnosis, prognosis, stratification and/or monitoring of a therapy, of a cancer disease in a subject.

In summary, the present invention provides a new method for the diagnosis, prognosis, stratification and/or monitoring of a therapy, of a cancer disease in a patient. The method is based on the determination of the level of CD3+ Tumor-Infiltrating Lymphocytes and CD24 expression. The new biomarker combination of the present invention allows diagnosing, prognosing, stratifying and/or monitoring of a therapy of various cancer diseases. Furthermore, the present application provides diagnostic kits for performing the methods of the invention.

1. A method for the diagnosis, prognosis, stratification and/or monitoring of a therapy, of a cancer disease in a subject, comprising the steps of:
(a) Providing a biological sample from the subject; and
(b) Determining the levels of at least two biomarkers in the biological sample, wherein the at least two biomarkers are CD3+ tumor-infiltrating lymphocytes (TIL) and CD24+ cells;
wherein levels of the at least two biomarkers in the biological sample from the subject as determined in step (b) that are different from a healthy control or reference values are indicative of the presence of a cancer disease in the subject.

2. The method according to claim 1, wherein the biological sample is a tissue sample or a body liquid sample.

3. The method according to claim 1, wherein the levels of the at least two biomarkers are assessed by their protein biomarkers, and wherein the determining the level of each of the at least two biomarkers involve determining the levels of proteins of the at least two biomarkers in the biological sample.

4. The method according to claim 1, wherein the method further comprises determining the level of one or more additional biomarkers in the biological sample, wherein the one or more additional biomarkers are selected from CD44 and markers for epithelial-mesenchymal transition.

5. The method according to claim 1, wherein the method is a screening method for establishing a first diagnosis of cancer in the subject.

6. The method according to claim 1, wherein if the determining of the levels of the at least two biomarkers in the biological sample results in a CD24+^{high}/CD3+ TIL^{low} phenotype, the subject is prognosed with a high risk of disease progression; and
wherein, when the subject is prognosed with a high risk of disease progression, a treatment plan for said patient is modified.

7. The method according to claim 1, wherein the cancer disease is a cancer selected from the group consisting of nasopharyngeal cancer, head and neck cancer, hepatocellular cancer, breast cancer, triple-negative breast cancer, colorectal cancer, gastric cancer, lymphomas including non-Hodgkin lymphomas, gliomas, melanomas, small-cell and non-small cell lung cancer, cholangiocarcinoma, renal cell cancer, bladder cancer, uterine cancer, epithelial ovarian cancer, prostate cancer, and pancreatic cancer.

8. The method according to claim 1, wherein the biomarker is detected using one or more binding agents or antigen-binding peptides.

9. The method according to claim 1, wherein one or more of the least two biomarkers is detected by immunohistochemistry by a method comprising the steps of:
(i) Providing a formalin-fixed paraffin-embedded (FFPE) tissue sample derived from the subject;
(ii) Dewaxing the FFPE tissue sample in xylene and rehydrating it using alcohol/water gradients;
(iii) Performing antigen retrieval using an antigen retrieval solution in a decloaking chamber pressure cooker;
(iv) Optionally, quenching endogenous peroxidase and/or blocking endogenous biotin;
(v) Incubating the sample with one or more primary antibodies that are specific for the one or more of the at least two biomarkers;
(vi) Incubating the sample using one or more secondary antibodies that are specific for either of said one or more primary antibodies, wherein said one or more secondary antibodies are conjugated with a reporter enzyme; wherein, optionally, if more than one secondary antibody is used, incubating the sample with the one or more secondary antibodies may be performed either simultaneously or subsequently; and
(vii) Detection of the reporter enzyme conjugated to said secondary antibody, wherein, optionally, if more than one primary antibody and/or more than one secondary antibody is used, detection of said secondary antibodies may be performed either simultaneously or subsequently.

10. The method according to claim 9, wherein the method further comprises one or more washing steps using a washing buffer between steps (iv) and (v); and/or between steps (v) and (vi); and/or between steps (vi) and (vii); and/or between the detection of the different secondary antibodies in step (vii); and/or
wherein the antigen retrieval solution of step (iii) is Tris-EDTA with a pH of about 9; and/or
wherein antigen retrieval in step (iii) is performed at a temperature in a range of about 90° C. to about 150° C., for a time period in a range of about 1 min to 15 min.

11. The method according to claim 1, wherein CD24 is detected using a binding agent or antigen-binding peptide that specifically binds to CD24, wherein the CD24 comprises an amino acid sequence which is at least 90% identical to SEQ ID No. 1;
wherein said binding agent or antigen-binding peptide binds to a sequence according to positions 46-48 of SEQ ID No. 1; and/or
wherein the binding agent or antigen-binding peptide that specifically binds to CD24 is selected from a group consisting of anti-CD24 monoclonal antibody clone: ML5, anti-CD24 monoclonal antibody clone: M1/69, anti-CD24 monoclonal antibody clone: SN3b, anti-CD24 monoclonal antibody clone: SN3, anti-CD24 monoclonal antibody clone: SWA11, and anti-CD24 monoclonal antibody clone: ALB9.

12. The method according to claim 1, wherein said subject is a mouse, a rat, a guinea pig, a rabbit, a cat, a dog, a monkey, or a human.

13. A method for evaluating the treatment success of a patient suffering from a cancer disease who received a cancer treatment, comprising the steps of:
(a) Providing a biological sample from the subject;
(b) Determining the levels of at least two biomarkers in the biological sample, wherein the at least two biomarkers are CD3+ tumor-infiltrating lymphocytes (TIL) and CD24+ cells; and
(c) Comparing the levels of said at least two biomarkers as determined in (b) with a reference sample or reference values,
wherein a decrease or an increase of the levels of the at least two biomarkers in the biological sample from said subject compared to said reference sample or reference values are indicative of the patient's response to said treatment.

14. The method according to claim 13, wherein said reference values correspond to the levels of the at least two biomarkers in a provided biological sample obtained from said patient before receiving said treatment; and/or
wherein if the CD24 level is high and/or the level of CD3+ TIL is low indicates an insufficient response by the patient to said treatment.

15. The method according to claim 13, wherein the cancer disease is locally advanced nasopharyngeal cancer.

16. The method according to claim 13, wherein the binding agent or antigen-binding peptide that specifically binds to CD24 is anti-CD24 monoclonal antibody clone: ML5.

17. The method according to claim 13, wherein the cancer disease is a cancer selected from the group consisting of nasopharyngeal cancer, head and neck cancer, hepatocellular cancer, breast cancer, triple-negative breast cancer, colorectal cancer, gastric cancer, lymphomas including non-Hodgkin lymphomas, gliomas, melanomas, small-cell and non-small cell lung cancer, cholangiocarcinoma, renal cell cancer, bladder cancer, uterine cancer, epithelial ovarian cancer, prostate cancer, and pancreatic cancer.

18. The method according to claim 13, wherein one or more of the least two biomarkers is detected by immunohistochemistry by a method comprising the steps of:
(i) Providing a formalin-fixed paraffin-embedded (FFPE) tissue sample derived from the subject;
(ii) Dewaxing the FFPE tissue sample in xylene and rehydrating it using alcohol/water gradients;
(iii) Performing antigen retrieval using an antigen retrieval solution in a decloaking chamber pressure cooker;
(iv) Optionally, quenching endogenous peroxidase and/or blocking endogenous biotin;
(v) Incubating the sample with one or more primary antibodies that are specific for the one or more of the at least two biomarkers;
(vi) Incubating the sample using one or more secondary antibodies that are specific for either of said one or more primary antibodies, wherein said one or more secondary antibodies are conjugated with a reporter enzyme; wherein, optionally, if more than one secondary antibody is used, incubating the sample with the one or more secondary antibodies may be performed either simultaneously or subsequently; and
(vii) Detection of the reporter enzyme conjugated to said secondary antibody, wherein, optionally, if more than one primary antibody and/or more than one secondary antibody is used, detection of said secondary antibodies may be performed either simultaneously or subsequently.

19. The method according to claim 13, wherein CD24 is detected using a binding agent or antigen-binding peptide that specifically binds to CD24, wherein the CD24 comprises an amino acid sequence which is at least 90% identical to SEQ ID No. 1;
wherein said binding agent or antigen-binding peptide binds to a sequence according to positions 46-48 of SEQ ID No. 1; and/or
wherein the binding agent or antigen-binding peptide that specifically binds to CD24 is selected from a group consisting of anti-CD24 monoclonal antibody clone: ML5, anti-CD24 monoclonal antibody clone: M1/69, anti-CD24 monoclonal antibody clone: SN3b, anti-CD24 monoclonal antibody clone: SN3, anti-CD24 monoclonal antibody clone: SWA11, and anti-CD24 monoclonal antibody clone: ALB9.

20. A diagnostic kit for performing a method according to claim 1, the kit comprising a combination of at least two antibodies, derivatives, or antigenic fragments, thereof, for the detection of any of CD24 and CD3;
wherein the diagnostic kit further comprises one or more components selected from a group consisting of visualization systems, blocking agents, chromogenic solutions, wash buffers, instructions for suitable operational parameters, secondary antibodies optionally conjugated with reporter enzymes, wherein said secondary antibodies are specific for at least one of said at least two antibodies, antigen retrieving solutions, detection kits for the detection of said conjugated reporter enzymes, and standard or control information so that the test sample can be compared with the control information standard.


SEQUENCE LISTING (1 sequence)

SEQ ID NO: 1 - protein, 80 aa, synthetic construct
MGRAMVARLGLGLLLLALLLPTQIYSSETTTGTSSNSSQSTSNSGLAPNPTNATTKAAGGALQSTASLFVVSLSLLHLYS